(19) United States
(12) Patent Application Publication
(10) Pub. No.: US 2023/0096051 A1
(43) Pub. Date: Mar. 30, 2023

(54) CGRP ANTIGONISTS USEFUL AS TRACER COMPOUNDS FOR POSITRON EMISSION TOMOGRAPHY

(71) Applicant: Eli Lilly and Company, Indianapolis, IN (US)

(72) Inventors: Mark Donald CHAPPELL, Indianapolis, IN (US); Miles Goodman SIEGEL, Indianapolis, IN (US)

(21) Appl. No.: 17/784,215

(22) PCT Filed: Dec. 4, 2020
(86) PCT No.: PCT/US2020/063401

§ 371 Date: Jun. 10, 2022

Related U.S. Application Data
(60) Provisional application No. 62/947,043, filed on Dec. 12, 2019.

(51) Int. Cl. C07D 401/14; C07B 59/00; A61K 51/04

(52) U.S. Cl. CPC C07D 401/14; C07B 59/002; A61K 51/0455; C07B 2200/05

(57) ABSTRACT

The present invention provides a compound of Formula I: wherein R¹ is hydrogen, F, or ¹⁸F; and R² is hydrogen, F, or ¹⁸F; or a pharmaceutically acceptable salt thereof, provided that when R¹ is ¹⁸F then R² is not ¹⁸F, useful as a CGRP receptor antagonist for PET imaging.

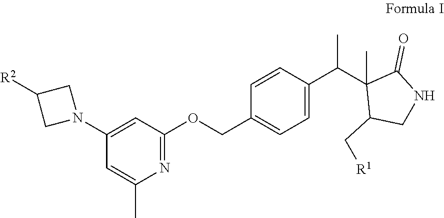

The present invention relates to certain novel calcitonin gene-related peptide (CGRP) receptor antagonist compounds useful as tracer compounds for positron emission tomography (PET) imaging of CGRP receptors, including diagnostic imaging, to pharmaceutical compositions comprising the compounds, to methods of using certain novel CGRP receptor antagonist compounds to prevent or treat certain physiological disorders such as migraine, and to intermediates and processes useful in the synthesis of the compounds.

The CGRP receptor PET tracer [11C]MK-4232 has been used to evaluate CGRP receptor occupancy of the CGRP antagonist telcagepant. (See S. G. G. Vermeersch, et al., The Journal of Headache and Pain, 14(suppl 1), page 224 (2013)). New PET tracers for imaging the CGRP receptor are desired, in particular, those which are blood brain barrier (BBB) permeable and which are less susceptible to active transport out of the central nervous system (CNS) by the P-glycoprotein (P-gp) efflux pump.

I. M. Bell, et al., Medicinal Chemistry Letters, 4, page 863-868 (2013) describes MK-4232 as the first PET tracer for the CGRP receptor. U.S. Pat. Nos. 9,637,495 and 9,708,297 each disclose certain CGRP receptor antagonist compounds useful in the treatment or prevention of migraine.

The present invention provides certain novel compounds that are antagonists of the CGRP receptor. In addition, the present invention provides certain novel radiolabeled compounds that are useful as PET tracers for imaging the CGRP receptor.

Accordingly, the present invention provides a compound of Formula I:

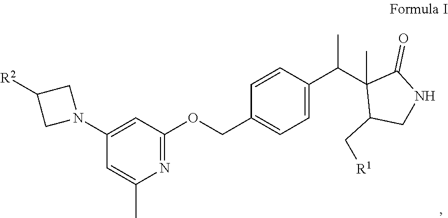

wherein R¹ is hydrogen, F, or ¹⁸F; and
R² is hydrogen, F, or ¹⁸F;
or a pharmaceutically acceptable salt thereof;
provided that when R¹ is ¹⁸F then R² is not ¹⁸F.

The present invention further provides a compound of Formula Ia:

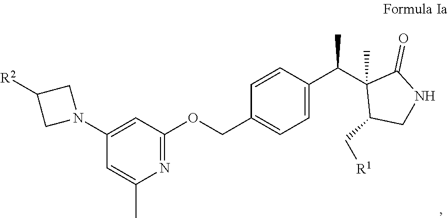

wherein R¹ is hydrogen, F, or ¹⁸F; and
R² is hydrogen, F, or ¹⁸F;
or a pharmaceutically acceptable salt thereof;
provided that when R¹ is ¹⁸F then R² is not ¹⁸F.

The present invention further provides a radiolabeled compound of Formula Ib:

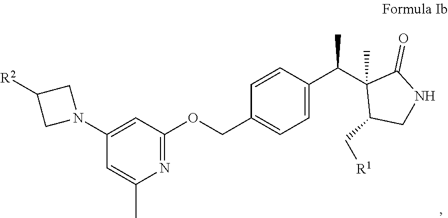

wherein R¹ is hydrogen or ¹⁸F; and
R² is hydrogen or ¹⁸F;
or a pharmaceutically acceptable salt thereof;
provided that when R¹ is ¹⁸F then R² is not ¹⁸F.

The present invention further provides a method of using a radiolabeled compound of Formula Ib

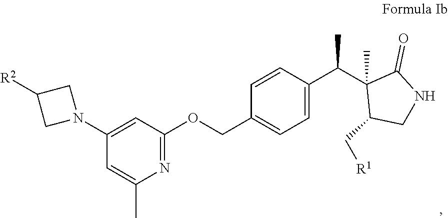

wherein R¹ is hydrogen or ¹⁸F; and
R² is hydrogen or ¹⁸F;
or a pharmaceutically acceptable salt thereof, provided that when R¹ is ¹⁸F then R² is not ¹⁸F, comprising introducing into a mammal a detectable quantity of the radiolabeled compound of Formula Ib, allowing sufficient time for the compound to become associated with CGRP receptors in the brain of the mammal, and then detecting the radiolabeled compound of Formula Ib in the brain of the mammal.

The present invention provides a method of preparing a radiolabeled compound of Formula Ib:

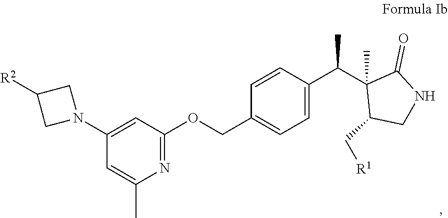

wherein R¹ is hydrogen or ¹⁸F; and
R² is hydrogen or ¹⁸F, provided that when R¹ is ¹⁸F then R² is not ¹⁸F,
comprising reacting a compound of Formula II with a source of [¹⁸F]fluoride:

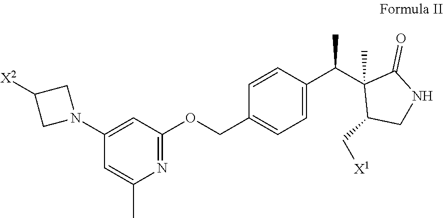

wherein X¹ is hydrogen or a suitable leaving group; or
X² is hydrogen or a suitable leaving group.

The present invention further provides an intermediate of Formula IIa:

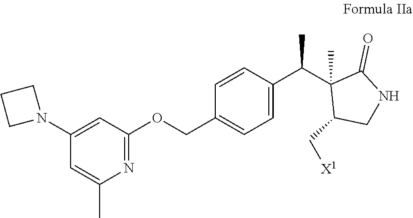

wherein X¹ is a suitable leaving group.

The present invention further provides an intermediate of Formula IIb:

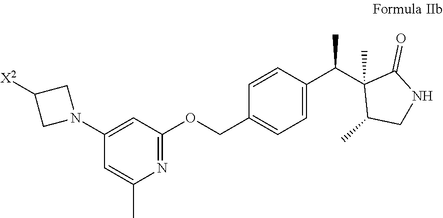

wherein X² is a suitable leaving group.

In addition, the present invention provides an intermediate of Formula IIc:

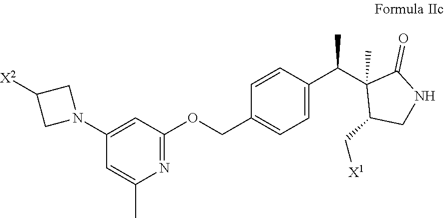

wherein X¹ and X² are each independently a suitable leaving group.

The present invention also provides a method of preventing migraine in a patient, comprising administering to a patient in need thereof an effective amount of a compound of Formula I or Formula Ia, or a pharmaceutically acceptable salt thereof.

The present invention further provides a method of treating migraine in a patient, comprising administering to a patient in need thereof an effective amount of a compound of Formula I or Formula Ia, or a pharmaceutically acceptable salt thereof. The present invention also provides a method of antagonizing the CGRP receptor in a patient, comprising administering to a patient in need thereof an effective amount of a compound of Formula I or Formula Ia, or a pharmaceutically acceptable salt thereof.

Furthermore, this invention provides a compound of Formula I or Formula Ia, or a pharmaceutically acceptable salt thereof for use in therapy, in particular for the treatment of migraine. In addition, this invention provides a compound of Formula I or Formula Ia, or a pharmaceutically acceptable salt thereof for use in preventing migraine. Even furthermore, this invention provides the use of a compound of Formula I or Formula Ia, or a pharmaceutically acceptable salt thereof, for the manufacture of a medicament for the treatment of migraine or for preventing migraine.

The invention further provides a pharmaceutical composition, comprising a compound of Formulas I, Ia, or Ib, or a pharmaceutically acceptable salt thereof, with one or more pharmaceutically acceptable carriers, diluents, or excipients. The invention further provides a process for preparing a pharmaceutical composition, comprising admixing a compound of Formulas I, Ia, or Ib, or a pharmaceutically acceptable salt thereof, with one or more pharmaceutically acceptable carriers, diluents, or excipients.

As used herein, the terms “treating”, “treatment”, or “to treat” includes restraining, slowing, stopping, or reversing the progression or severity of an existing symptom or disorder.

As used herein, the term “preventing” or “prevention” refers to protecting a patient who is prone to a certain disease or disorder, such as migraine, but is not currently suffering from symptoms of the disease or disorder, such as symptoms of migraine.

As used herein, the term “patient” refers to a mammal, in particular a human.

The preferred method of detecting the radiolabeled compound in the brain of the mammal is positron emission tomography.

As used herein, the term “effective amount” refers to the amount or dose of compound of the invention, or a pharmaceutically acceptable salt thereof which, upon single or multiple dose administration to the patient, provides the desired effect in the patient under diagnosis or treatment.

An effective amount may be readily determined by one skilled in the art by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

Compounds of the present invention are effective at a dosage per day that falls within the range of about 0.01 to about 20 mg/kg of body weight. In some instances dosage levels below the lower limit of the aforesaid range may be more than adequate, while in other cases, still larger doses may be employed with acceptable side effects, and therefore the above dosage range is not intended to limit the scope of the invention in any way.

The compounds of the present invention are formulated as pharmaceutical compositions administered by any route which makes the compound bioavailable. Such pharmaceutical compositions and processes for preparing same are well known in the art (See, e.g., Remington: The Science and Practice of Pharmacy, L. V. Allen, Editor, 22^{nd} Edition, Pharmaceutical Press, 2012).

The compound of Formula I wherein the methyl and the —CH₂R¹ substituents on the pyrrolidine ring are in the cis configuration, or pharmaceutically acceptable salt thereof, is preferred. For example, one of ordinary skill in the art will appreciate that the methyl substituent at position 3 is in the cis configuration relative to the —CH₂R¹ substituent at position 4 as shown in Scheme A below:

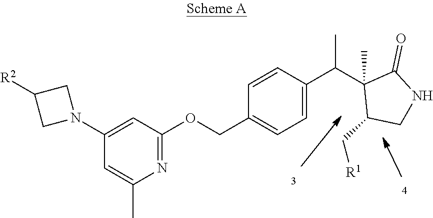

The following compounds are also preferred:

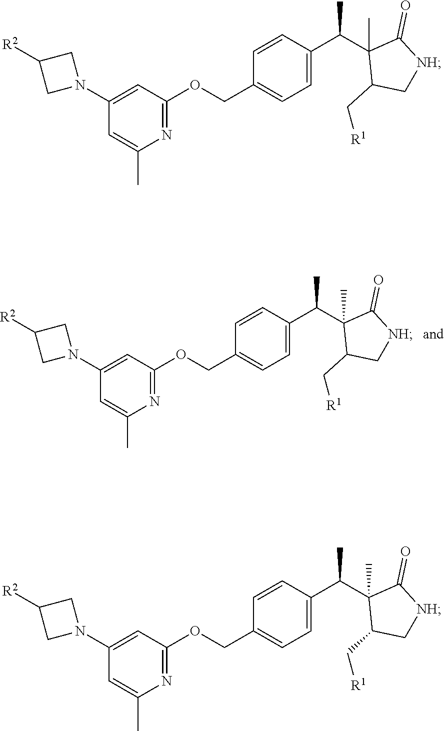

wherein R¹ is hydrogen, F, or ¹⁸F; and
R² is hydrogen, F, or ¹⁸F;
and the pharmaceutically acceptable salts thereof;
provided that when R¹ is ¹⁸F then R² is not ¹⁸F.

The following compounds are particularly preferred:

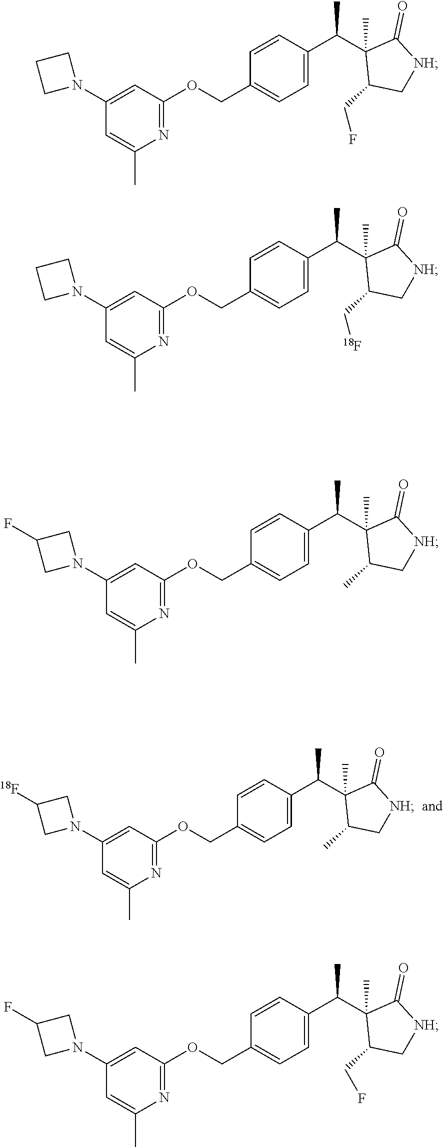 [SMILES: Cc1cc(N2CCC2)cc(OCc2ccc([C@@H](C)[C@@]3(C)C(=O)NC[C@@H]3CF)cc2)n1 ; Cc1cc(N2CCC2)cc(OCc2ccc([C@@H](C)[C@@]3(C)C(=O)NC[C@@H]3C[18F])cc2)n1 ; Cc1cc(N2CC(F)C2)cc(OCc2ccc([C@@H](C)[C@@]3(C)C(=O)NC[C@@H]3C)cc2)n1 ; Cc1cc(N2CC([18F])C2)cc(OCc2ccc([C@@H](C)[C@@]3(C)C(=O)NC[C@@H]3C)cc2)n1 ; "and" ; Cc1cc(N2CC(F)C2)cc(OCc2ccc([C@@H](C)[C@@]3(C)C(=O)NC[C@@H]3CF)cc2)n1]

and the pharmaceutically acceptable salts thereof.

Certain intermediates described in the following preparations may contain one or more nitrogen protecting groups. It is understood that protecting groups may be varied as appreciated by one of skill in the art depending on the particular reaction conditions and the particular transformations to be performed. The protection and deprotection conditions are well known to the skilled artisan and are described in the literature (See for example “Greene's Protective Groups in Organic Synthesis”, Fourth Edition, by Peter G. M. Wuts and Theodora W. Greene, John Wiley and Sons, Inc. 2007).

Individual isomers, enantiomers, and diastereomers may be separated or resolved by one of ordinary skill in the art at any convenient point in the synthesis of compounds of the invention, by methods such as selective crystallization techniques or chiral chromatography (See, for example, J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994).

A pharmaceutically acceptable salt of the compounds of the invention can be formed, for example, by reaction of an appropriate free base of a compound of the invention, with an appropriate pharmaceutically acceptable acid in a suitable solvent such as diethyl ether under standard conditions well known in the art. Additionally, the formation of such salts can occur simultaneously upon deprotection of a nitrogen protecting group. The formation of such salts is well known and appreciated in the art. See, for example, Gould, P. L., “Salt selection for basic drugs,” International Journal of Pharmaceutics, 33: 201-217 (1986); Bastin, R. J., et al. “Salt Selection and Optimization Procedures for Pharmaceutical New Chemical Entities,” Organic Process Research and Development, 4: 427-435 (2000); and Berge, S. M., et al., “Pharmaceutical Salts,” Journal of Pharmaceutical Sciences, 66: 1-19, (1977).

Certain abbreviations are defined as follows: “ACN” refers to acetonitrile; “BEH” refers to Ethylene Bridged Hybrid particle technology for HPLC particle sizes; “Ci” refers to Curie or Curies; “conc” refers to concentration; “c-Pr” refers to cyclopropyl; “DBU” refers to 1,8-diazabicyclo(5.4.0)undec-7-ene; “DCM” refers to DCM or methylene chloride; “DMEA” refers to N,N-dimethylethylamine; “DIPEA” refers to N,N-diisopropylethylamine; “DMF” refers to N,N-dimethylformamide; “DMAP” refers to 4-dimethylaminopyridine; “DMSO” refers to dimethylsulfoxide; “EDTA” refers to ethylenediaminetetraacetic acid; “EOS” refers to End of Synthesis; “ES/MS” refers to Electrospray Mass Spectrometry; “Et” refers to ethyl; “Et₂O” refers to diethyl ether; “EtOAc” refers to ethyl acetate; “EtOH” refers to ethanol; “TFA” refers to trifluoroacetic acid; “g” when used in reference to centrifugation, refers to relative centrifugal force; “HPLC” refers to High Performance Liquid Chromatography; “HP-BCD” refers to 20-hydroxypropyl-beta-cyclodextrin; “h” or “hr” refers to hour or hours as a unit of time; “HTRF” refers to Homogeneous Time Resolved Fluorescence; “IC₅₀” refers to the concentration of an agent that produces 50% of the maximal inhibitory response possible for that agent; “kPa” refers to kilopascal or kilopascals; “kV” refers to kilovolts; “LC-ES/MS” refers to Liquid Chromatography Electrospray Mass Spectrometry; “LC-MS/MS” refers to Liquid chromatography-tandem mass spectrometry; “LDA” refers to lithium diisopropylamide; “LG” refers to leaving group; “mA” refers to milliamps or milliamperes; “MDCK” refers to Madin-Darby canine kidney epithelial cells; “min” refers to minute or minutes as a unit of time; “Me” refers to methyl; “MeOH” refers to methanol or methyl alcohol; “Ms” refers to mesyl, or methylsulfonyl or methanesulfonyl; “MTBE” refers to methyl-tert-butyl ether; “NaHMDS” refers to sodium bis(trimethylsilyl) amide; “n-BuLi” refers to n-butyllithium; “ng” refers to nanogram or nanograms; “PW” refers to purified water; “OAc” refers to acetate; “OMs” refers to mesylate; “OTs” refers to tosylate; “psi” refers to pounds per square inch; “rpm” refers to revolutions per minute; “RT” refers to room temperature; “SD” refers to Standard Deviation; “sec” refers to second or seconds as a unit of time; “SEM” refers to standard error of the mean; “TBAF” refers to tetrabutylammonium fluoride; “t-BuOH” refers to tert-butanol; “TEA” refers to triethylamine; “Ts” refers to tosyl, or 4-methylbenzenesulfonyl; “TFA” refers to trifluoroacetic acid; “THF” refers to tetrahydrofuran; “TMEDA” refers to tetrametylethylenediamine; “tR” refers to retention time; “Tris-HCl” refers to tris(hydroxymethyl)aminomethane hydrochloride; “U/mL” refers to units per milliliter; “WFI” refers to water for injection.

The compounds of the present invention, or salts thereof, may be prepared by a variety of procedures known to one of ordinary skill in the art, some of which are illustrated in the schemes, preparations, and examples below. One of ordinary skill in the art recognizes that the specific synthetic steps for each of the routes described may be combined in different ways, or in conjunction with steps from different schemes, to prepare compounds of the invention, or salts thereof. The products of each step in the schemes below can be recovered by conventional methods well known in the art, including extraction, evaporation, precipitation, chromatography, filtration, trituration, and crystallization. In the schemes below, all substituents unless otherwise indicated, are as previously defined. The reagents and starting materials are readily available to one of ordinary skill in the art. The following schemes, preparations, examples, and assays further illustrate the invention, but should not be construed to limit the scope of the invention in any way.

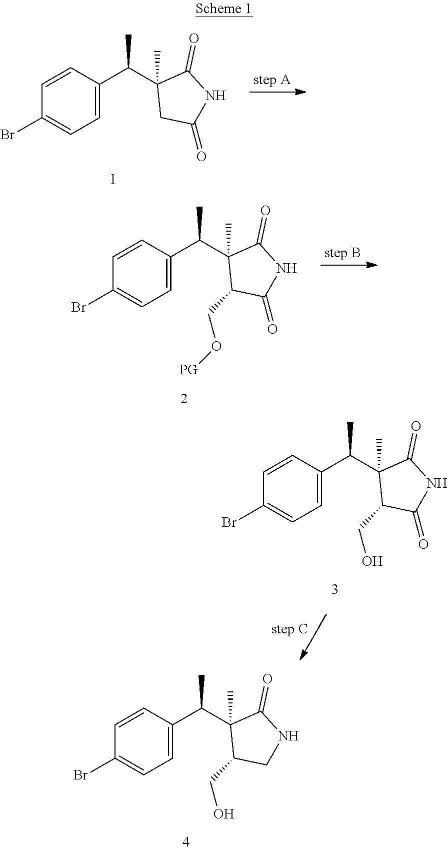

In Scheme 1, step A, one skilled in the art will recognize that (3R,4R)-3-((R)-1-(4-bromophenyl)ethyl)-3,4-dimethylpyrrolidine-2,5-dione (1, U.S. Pat. No. 9,708,297, published Feb. 2, 2017) may be doubly deprotonated with a suitably strong base and treated with an electrophile such as an alkyl halide to obtain the alkylated pyrrolidinone 2. For example, compound 1 may be treated with 2 or more equivalents of an organolithium reagent at about −78° C. to about RT in a suitable polar aprotic solvent, such as THF or 1,4-dioxane. The resulting dianion may be treated with about 1 or more equivalents of a wide array of desired electrophiles, such as a suitably protected alkoxy halide, mesylate, or tosylate, such as methoxymethyl chloride, t-butoxymethyl chloride, a trialkylsilylethoxymethyl halide or tosylate, or (substituted) benzyloxymethyl chloride or tosylate, among others. More specifically, about 1 equivalent of compound 1 may be treated with about 2.3 equivalents of LDA at about −10° C. in THF, and the dianion may be captured by the addition of about 1.2 equivalents of 2-(trimethylsilyl)ethoxymethyl chloride, with subsequent quenching with water. The product can then be isolated and purified utilizing techniques well known in the art, such as extraction methods, using, for example, EtOAc, MTBE, Et₂O, or DCM, to provide compound 2 (PG=-(trialkylsilyl)ethyl).

In Scheme 1, step B, the (trialkylsilyl)ethyl protecting group may be cleaved under an array of conditions well known in the art, such as with AcOH, TFA, or TBAF in a suitable organic solvent. For example, about 1 equivalent of compound 2 may be treated with excess TFA in DCM at about RT. The product can then be isolated and purified utilizing techniques well known in the art, such as extraction methods, using, for example, EtOAc, MTBE, Et₂O, or DCM, to provide compound 3.

In Scheme 1, step C, one skilled in the art will recognize the possibility of regioselective reduction of the succinimide carbonyl using an array of reducing agents, such as with a metal hydride, borohydride salt, or diborane in a polar aprotic solvent. More specifically, compound 3 may be treated slowly with about 1 equivalent of NaBH₄ at about 0° C. to RT, with an acid quench of the reaction mixture. The product can then be isolated and purified utilizing techniques well known in the art, such as extraction methods, using, for example, DCM and MeOH, and chromatography, to obtain 4.

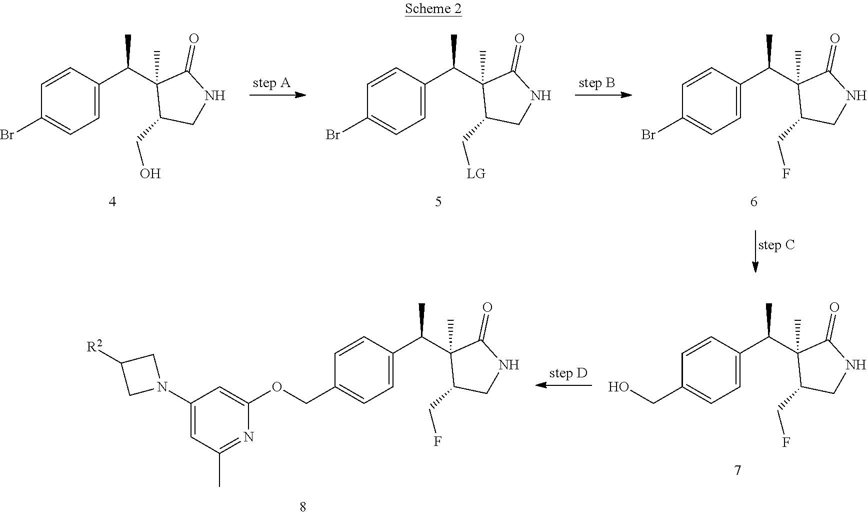

In Scheme 2, step A, one skilled in the art will recognize that the pendant hydroxyl group may be converted to one of many suitable leaving groups (e.g., LG=Cl, Br, OSO₂CH₃, OSO₂Ph, among others well known in the art) under a wide array of conditions as well described in the art. For example, compound 4 may be treated with of a non-nucleophilic organic base at about −78° C. to RT in an organic solvent such as DCM, with subsequent treatment of an alkyl- or aryl-sulfonyl chloride. More specifically, about 1 equivalent of compound 4 may be treated with about 2 equivalents of TEA in DCM at about RT, and the resulting mixture may be treated dropwise with about 1.1 equivalents of methanesulfonyl chloride. The product can then be isolated and purified utilizing techniques well known in the art, such as extraction methods, using, for example, EtOAc, MTBE, Et₂O, or DCM, to provide compound 5 (LG=—OSO₂CH₃).

In Scheme 2, step B, the mesylate of 5 may be displaced by fluoride anion in an S_{N}2-type reaction under a wide array of conditions well known in the art. For example, compound 4 may be dissolved in a suitable polar solvent and irradiated in a microwave in the presence of a fluoride source. More specifically, about 1 equivalent of 5 and about 1.6 equivalents of CsF may be placed in IPA and irradiated at about 130° C. in a microwave for about 3 h. The product can then be isolated and purified utilizing techniques well known in the art, such as extraction methods, using, for example, EtOAc, MTBE, Et₂O, or DCM, and column chromatography on silica gel, to obtain compound 6.

In Scheme 2, step C, one skilled in the art will recognize that the bromide in 6 may be carbonylated under various conditions, including transition-metal mediated processes under an atmosphere of carbon monoxide, or lithium-halogen exchange with in situ quenching of the aryllithium species using, for example, carbon monoxide or DMF.

The aldehyde intermediate generated may be isolated and purified if stable, or may be reduced in situ under standard reduction conditions. More specifically, about 1 equivalent of the bromide 6 may be heated with about 0.03-0.2 equivalents Pd(OAc)₂ and about 0.1-0.2 equivalents of a suitable phosphine ligand, such as butyldi(1-adamantyl)phosphine, in the presence of about 1.1 equivalents of a suitable bidentate, non-nucleophilic base, such as TMEDA, under an atmosphere of carbon monoxide/hydrogen at about 65 psi at about 95° C. overnight. The reaction mixture may be cooled to RT and the crude aldehyde product of the palladium-mediated reaction may be isolated and purified utilizing techniques well known in the art, such as extraction methods, using, for example, EtOAc, MTBE, Et₂O, or DCM, and column chromatography on silica gel. Subsequent reduction may be performed with about 1.2-1.5 equivalents NaBH₄ in a polar organic solvent, such as EtOH, at about 0° C. The product can then be isolated and purified utilizing techniques well known in the art, such as extraction methods, using, for example, EtOAc or DCM, and reverse-phase chromatography on C18 silica gel, to obtain compound 7.

In Scheme 2, step D, 7 may be arylated with an appropriately substituted 2-halopyridine under well-known S_{N}Ar conditions with heating and microwave irradiation, or, more preferably, by transition-metal-mediated Ullman or Buchwald-Hartwig etherification conditions as described in the literature (B. Liu, B.-F. Shi, Tet. Lett 56 (1), Jan. 1, 2015, pp. 15-22). The skilled artisan will recognize that the requisite 6-methyl-4-substituted aminopyridine needed in this etherification step may be prepared from 2,4-dichloro-6-methylpyridine or 4-bromo-2-chloro-6-methylpyridine and an appropriately substituted amine under, for example, copper-mediated Ullmann-coupling conditions or palladium-mediated Buchwald-Hartwig coupling conditions, as are well described in the art. For example, about 1 equivalent of compound 7 and about 1.2 equivalents of an appropriately substituted (azetidin-1-yl)-2-chloro-6-methyl-pyridine may be heated under an atmosphere of N₂ in the presence of a palladium(0)-ligand-base mixture (1:10:240, prepared, for example, from a mixture of tris(dibenzylideneacetone) dipalladium(0), 2-(di-tert-butylphosphino)-2′,4′,6′-triisopropyl-3,6-dimethoxy-1,1′-biphenyl, and Cs₂CO₃), at about 85° C. for about 16-24 h. The product can then be isolated and purified utilizing techniques well known in the art, such as extraction methods, using, for example, EtOAc or DCM, and reverse-phase chromatography on C18 silica gel, to obtain the arylether compound 8 (R²═H, F).

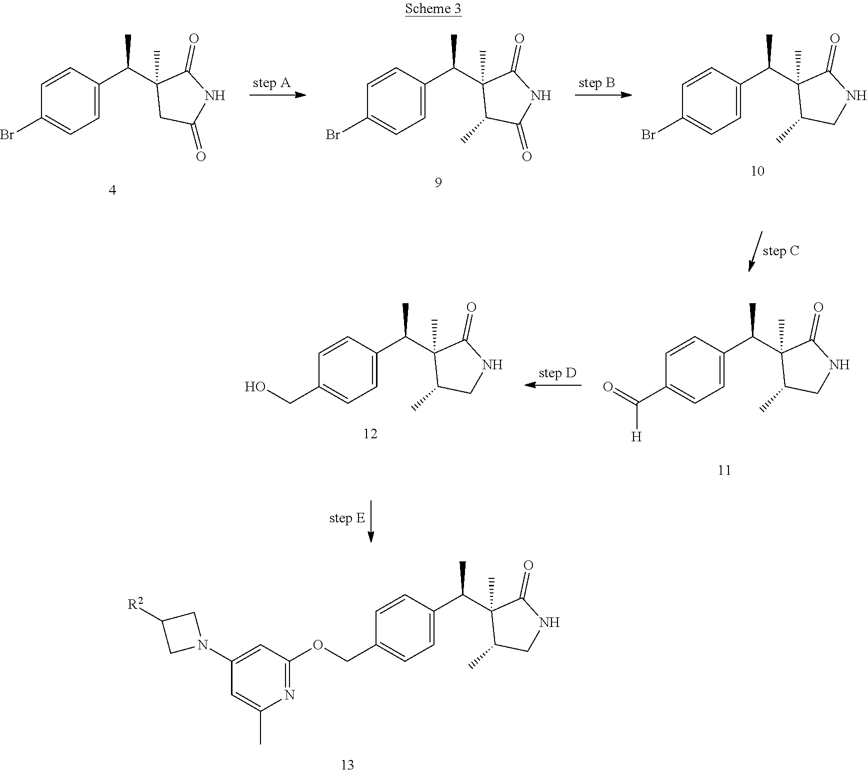

In Scheme 3, step A, compound 4 may be alkylated under a variety of conditions well known in the art, such as by treatment with an organic or inorganic base, for example, treatment with an alkoxide (such as sodium or potassium t-butoxide), methyl- or n-butyllithium, a Grignard reagent, or, more preferably, a base such as sodium or potassium hydride, lithium hexamethyldisilazide, or LDA, in a suitable organic solvent, such as THF or 1,4-dioxane, with subsequent treatment of the di-anion with a methylating agent such as a methyl halide. More specifically, about 1 equivalent of compound 4 may be treated with 2.2-3 equivalents of lithium bis(trimethylsilyl)amide in THF for about 1-2 hr at RT followed by slow addition about 0.8-1 equivalent of methyl iodide. The product can then be isolated and purified utilizing techniques well known in the art, such as extraction methods and chromatography, to provide compound 9.

In Scheme 3, step B, reduction of the succinimide carbonyl may be effected in a manner similar to that described in Scheme 1, step C. More specifically, about 1 equivalent of 9 may be treated with about 5 equivalents of borane dimethyl sulfide complex in a suitable polar organic solvent, such as THF or 1,4-dioxane, at 0° C. The reaction mixture may be quenched with a suitable protic solvent, such as MeOH, concentrated under reduced pressure, and the crude material may be treated with about 2 equivalents of a reducing agent, such as NaBH₄, in a protic solvent, such as TFA. The reduced product 10 may be isolated and purified utilizing techniques well known in the art, such as extraction methods and chromatography, to provide compound 10.

In Scheme 3, step C, carbonylation of compound 10 to 11 with subsequent reduction to the hydroxymethyl compound 12, as in Scheme 3, step D, may be effected in a manner similar to that described in Scheme 2, step C. Aldehyde 11 may be isolated and purified, utilizing techniques well known in the art, such as extraction methods and chromatography, or may be carried on directly to the reduction step D.

In Scheme 3, step E, aryl etherification of compound 12 may be effected in a manner similar to that described in Scheme 2, step D, to obtain the arylether compound 13. (R²═F). The skilled artisan will recognize that the requisite 6-methyl-4-substituted aminopyridine needed in this etherification step may be prepared from 2,4-dichloro methylpyridine or 4-bromo-2-chloro-6-methylpyridine and an appropriately substituted amine under, for example, copper-mediated Ullmann-coupling conditions or palladium-mediated Buchwald-Hartwig coupling conditions, as well described in the art.

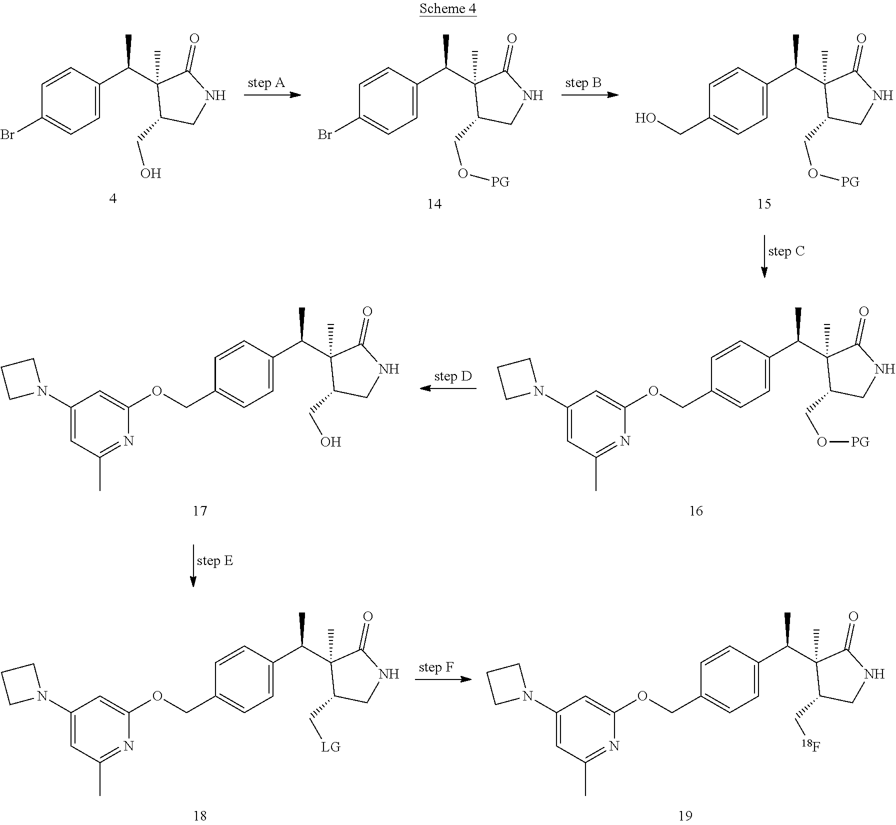

In Scheme 4, step A, the alcohol product 4 from Scheme 1, step C, may be protected using a variety of protecting groups well known in the art. For example, silyl ethers as alcohol protecting groups are especially widely used due to their ease of formation and removal, which can be modulated by both electronic and steric groups around the silicon atom, enabling deprotection under a variety of acidic or basic conditions as needed. More specifically, about 1 equivalent on alcohol 4 may be treated with about 1.5 equivalents of tert-butyldimethylchlorosilane in the presence of about 1.5 equivalents of a suitable base, such as TEA/DMAP, imidazole, or DBU, in a suitable aprotic solvent, such as DCM, THF, or 1,4-dioxane, from about 0° C. to reflux for 2-24 h. The product can then be isolated and purified utilizing techniques well known in the art, such as extraction methods and chromatography, to provide compound 14 (PG=tert-butyldimethylsilyl).

In Scheme 4, step B, carbonylation of the bromide in compound 12 with subsequent reduction to the hydroxymethyl compound 15 (e.g., PG=tert-butyldimethylsilyl) may be effected in a manner similar to that described in Scheme 2, step C.

In Scheme 4, step C, aryl etherification of compound 15 (e.g., PG=tert-butyldimethylsilyl) may be effected in a manner similar to that described in Scheme 2, step D, to obtain the arylether compound 16 (e.g., PG=tert-butyldimethylsilyl). The skilled artisan will recognize that the requisite 4-(azetidin-1-yl)-6-methyl-4-substituted aminopyridine needed in this etherification step may be prepared, for example, from 2,4-dichloro-6-methylpyridine and azetidine via copper-mediated Ullmann coupling conditions or palladium-mediated Buchwald-Hartwig coupling conditions, as well described in the art.

In Scheme 4, step D, deprotection of compound 16 (e.g., PG=tert-butyldimethylsilyl) to the alcohol 17 may be accomplished utilizing one of myriad deprotection conditions, depending on the protecting group. For example, when the protecting group is a silyl ether, one of many fluoride sources, such as TBAF, NH₄F, or KF, in a suitable polar solvent such as THF or 1,4-dioxane, may be used. More specifically, about 1 equivalent of the protected compound 16 (e.g., PG=tert-butyldimethylsilyl) may be treated with a portion wise excess of a solution of TBAF in THF at RT for 18-24 h. The product can then be isolated and purified utilizing techniques well known in the art, such as extraction methods and reverse-phase chromatography, to obtain the deprotected alcohol 17.

In Scheme 4, step E, the alcohol moiety of 17 may be converted to a suitable leaving group as described in Scheme 2, step A. More specifically, about 1 equivalent of the alcohol 17 may be treated with a portion wise excess of p-toluenesulfonyl chloride in the presence of an excess of a suitable amine, such as TEA or DIPEA, at RT for 6-24 h. The product can then be isolated and purified utilizing techniques well known in the art, such as extraction methods and chromatography, to obtain compound 18 (LG=OTs).

In Scheme 4, step F, compound 18 (LG=OTs or other suitable leaving group) may be displaced by [¹⁸F] fluoride under conditions well known in the art. For example, compound 18, where the leaving group is methanesulfonyl or 4-methylbenzenesulfonyl, may be treated with a suitable source of ¹⁸F (e.g., [¹⁸F]F—) in the presence of a suitable non-nucleophilic base such as K₂CO₃ in a suitable polar solvent such as DMSO to obtain the compound 19. Sources of ¹⁸F fluoride include [¹⁸F]FK₂₂₂. More specifically, about 1 mg of compound 18 dissolved in anhydrous DMSO may be added to a reaction vial containing anhydrous [¹⁸F]FK₂₂₂—K₂CO₃ (prepared from [¹⁸F]F, obtained from a cyclotron facility, trapped onto an ion exchange cartridge and eluted with a solution of Kryptofix 222/K₂CO₃ in ACN and evaporated under anhydrous conditions at about 100° C.) and heated for about 10 min at about 120° C. under an atmosphere of helium. The reaction mixture may be diluted with appropriate HPLC solvents, such as EtOH, ACN, and water, and the product may be purified utilizing techniques well known in the art, such as semi-preparative reverse phase column chromatography, to obtain the radiolabelled compound 19.

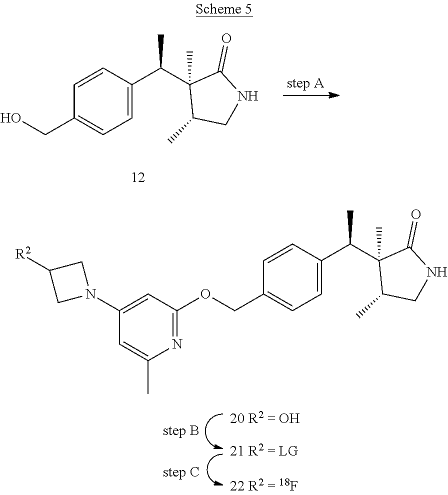

In Scheme 5, step A, aryl etherification of compound 12 may be effected in a manner similar to that described in Scheme 2, step D, to obtain the arylether compound 20 (R²═OH). The skilled artisan will recognize that the suitable leaving group may be one of many known in the art, such as tosylate, mesylate, chloride, bromide, and the like. Additionally, the skilled artisan will recognize the requisite 4-(azetidin-1-yl)-6-methyl-4-substituted aminopyridine needed in this etherification step may be prepared, for example, from 2,4-dichloro-6-methylpyridine and azetidine via copper-mediated Ullmann coupling conditions or palladium-mediated Buchwald-Hartwig coupling conditions well known in the art.

In Scheme 5, step B, the terminal hydroxy group of 20 may be converted to a suitable leaving group (e.g., LG=OTs, OMs, Cl) under conditions similar to those described for Scheme 2, step A, to obtain compound 21.

In Scheme 5, step C, the suitable leaving group of compound 21 may be displaced by [¹⁸F]fluoride under conditions similar to those described in Scheme 4, step F, to obtain compound 22 (R²=₁₈F).

### PREPARATIONS AND EXAMPLES

The following Preparations and Examples further illustrate the invention and represent typical synthesis of the compound of the invention. The reagents and starting materials are readily available or may be readily synthesized by one of ordinary skill in the art. It should be understood that the Preparations and Examples are set forth by way of illustration and not limitation, and that various modifications may be made by one of ordinary skill in the art.

The R- or S-configuration of the compound of the invention may be determined by standard techniques such as X-ray analysis and correlation with chiral-HPLC retention time.

LC-ES/MS is performed on an AGILENT® HP1100 liquid chromatography system. Electrospray mass spectrometry measurements (acquired in positive and/or negative mode) are performed on a Mass Selective Detector quadrupole mass spectrometer interfaced to the HP1100 HPLC. LC-MS conditions (low pH): column: PHENOMENEX® GEMINI® NX C18 2.1×50 mm 3.0 μm; gradient: 5-100% B in 3 min, then 100% B for 0.75 min column temperature: 50° C.+/−10° C.; flow rate: 1.2 mL/min; Solvent A: deionized water with 0.1% HCOOH; Solvent B: ACN with 0.1% formic acid; wavelength 214 nm. Alternate LC-MS conditions (high pH): column: XTERRA® MS C18 columns 2.1×50 mm, 3.5 μm; gradient: 5% of solvent A for 0.25 min, gradient from 5% to 100% of solvent B in 3 min and 100% of solvent B for 0.5 min or 10% to 100% of solvent B in 3 min and at 100% of solvent B for 0.75 min; column temperature: 50° C.+/−10° C.; flow rate: 1.2 mL/min; Solvent A: 10 mM NH₄HCO₃ pH 9; Solvent B: ACN; wavelength: 214 nm.

Preparative reversed phase chromatography is performed on an AGILENT® 1200 LC-ES/MS equipped with a Mass Selective Detector mass spectrometer and a LEAP® autosampler/fraction collector. High pH methods are run on a 75×30 mm PHENOMENEX® GEMINI®-NX, 5μ particle size column with a 10×20 mm guard. Flow rate of 85 mL/min. Eluent is 10 mM ammonium bicarbonate (pH 10) in acetonitrile unless noted otherwise.

NMR spectra are performed on a Bruker AVIII HD 400 MHz NMR Spectrometer, obtained as CDCl₃ or DMSO solutions reported in ppm, using residual solvent [CDCl₃, 7.26 ppm; (CD₃)₂SO, 2.05 ppm] as reference standard. When peak multiplicities are reported, the following abbreviations may be used: s (singlet), d (doublet), t (triplet), q (quartet), m (multiplet), br-s (broad singlet), dd (doublet of doublets), dt (doublet of triplets). Coupling constants (J), when reported, are reported in hertz (Hz).

### Preparation 1

### (3R,4S)-3-((R)-1-(4-bromophenyl)ethyl)-3-methyl-4-((2-(trimethylsilyl)ethoxy)methyl)pyrrolidine-2,5-dione

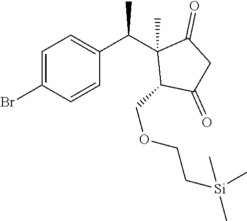 [SMILES: C[C@H](c1ccc(Br)cc1)[C@@]1(C)C(=O)CC(=O)[C@@H]1COCC[Si](C)(C)C]

Scheme 1, step A: To a stirred solution of DIPEA (110 mL, 782 mmol) in THF (1 L) at −13° C. is added a 2.5 M solution of n-BuLi in hexanes (310 mL, 780 mmol) and the resulting mixture is stirred for 1 h. To this solution is added (3R,4R)-3-((R)-1-(4-bromophenyl)ethyl)-3,4-dimethylpyrrolidine-2,5-dione (100 g, 338 mmol) dissolved in THF (200 mL) and the resulting mixture is stirred at −10° C. for 1 h. To the resulting mixture is added a solution of 2-(trimethylsilyl)ethoxymethyl chloride (70 mL, 376 mmol) and the reaction mixture is stirred at −10° C. for 2 h. The reaction is carefully quenched with water (750 mL), the pH is adjusted to ˜3.5 using aqueous 5 N HCl (˜220 mL), and the acidified mixture is extracted with MTBE (1 L) The organic phase is washed sequentially with water (2×500 mL) and saturated aqueous NaCl (500 mL) and concentrated under reduced pressure to give an orange oil. The crude material is purified by column chromatography on silica gel, eluting with 5 to 30% of EtOAc in hexanes. The pure chromatography fractions are combined and concentrated under reduced pressure to obtain the title compound (87.4 g, 61% yield). ¹H NMR (CDCl₃) δ 0.02 (s, 9H), 0.80-0.84 (m, 2H), 1.32, (s, 3H), 1.34 (d, J=7.1 Hz, 3H), 3.00 (m, 1H), 3.08 (q, J=7.1 Hz, 1H), 3.31-3.41 (m, 3H) 3.62 (m, 1H), 7.08 (d, J=8.4 Hz, 2H), 7.42 (d, J=8.4 Hz, 2H), 9.05 (s, 1H). ES/MS (m/z, for ⁷⁹Br, ⁸¹Br): 424.0, 426.0 (M+H).

### Preparation 2

### (3R,4S)-3-((R)-1-(4-bromophenyl)ethyl)-4-(hydroxymethyl)-3-methylpyrrolidine-2,5-dione

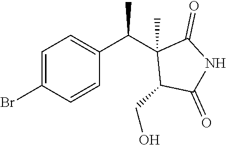 [SMILES: C[C@H](c1ccc(Br)cc1)[C@@]1(C)C(=O)NC(=O)[C@@H]1CO]

Scheme 1, step B: To a stirred solution of (3R,4S)-3-((R)-1-(4-bromophenyl)ethyl)-3-methyl-4-((2-(trimethylsilyl)ethoxy)methyl)pyrrolidine-2,5-dione (86.5 g, 203 mmol) in DCM (450 mL) at RT is carefully added TFA (110 mL, 1455 mmol) over 5 min. The reaction is stirred for 3 h and diluted with DCM (500 mL). The mixture is cooled to 5° C. prior to the addition of aqueous 5 N NaOH until basic pH (˜12). The mixture is diluted with water (600 mL). The phases are separated and the aqueous layer is acidified to pH˜3 with aqueous 5 N HCl. This acidified mixture is extracted with MTBE (300 mL). The organic phase is washed sequentially with aqueous saturated NaHCO₃ (100 mL) and saturated aqueous NaCl (50 mL), dried over anhydrous Na₂SO₄, filtered, and concentrated under reduced pressure to give the title compound as a foamy white solid (52.1 g, 79% yield). ¹H NMR (CDCl₃) δ 1.22, (s, 3H), 1.36 (d, J=7.2 Hz, 3H), 3.10 (dd, J₁=6.8 Hz, J₂=4.4 Hz, 1H), 3.15 (q, J=7.2 Hz, 1H), 3.52 (dd, J₁=11.2 Hz, J₂=4.8 Hz, 1H), 3.63 (dd, J₁=11.2 Hz, J₂=6.8 Hz, 1H), 7.12 (d, J=8.4 Hz, 2H), 7.48 (d, J=8.4 Hz, 2H), 8.58 (s, 1H). ES/MS (m/z for ⁷⁹Br, ⁸¹Br): 324.0, 326.0 (M+H).

### Preparation 3

### (3R,4R)-3-[(1R)-1-(4-bromophenyl)ethyl]-4-(hydroxymethyl)-3-methyl-pyrrolidin-2-one

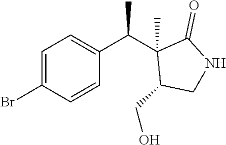 [SMILES: C[C@H](c1ccc(Br)cc1)[C@@]1(C)C(=O)NC[C@@H]1CO]

Scheme 1, step C: To a stirred solution of (3R,4S)-3-[(1R)-1-(4-bromophenyl)ethyl]-4-(hydroxymethyl)-3-methyl-pyrrolidine-2,5-dione (32 g, 98 mmol) in THF (320 mL) in a flask cooled in a cold water bath is carefully added NaBH₄ (11 g, 300 mmol) portion wise. The mixture is stirred at RT overnight. The reaction is cooled in an ice/water bath and carefully treated with TFA (160 mL) dropwise while maintaining the temperature above 25° C. After 30 minutes, the reaction mixture is treated with MeOH (160 mL) and water (160 mL) and stirred for 1 h. The mixture is concentrated under reduced pressure at 40° C. to yield a thick slurry, which is diluted with DCM (320 mL) and water (200 ml). The mixture is treated with aqueous 5 N HCl (100 ml). Water (100 ml) is added, followed by enough aqueous 5 N NaOH to reach pH˜14. The resulting emulsion is treated with MeOH (30 mL). The organic layer is separated, washed sequentially with water (100 mL), aqueous 5 N NaOH (10 ml) twice, and saturated aqueous NaCl (150 mL), dried over Na₂SO₄, filtered, and concentrated under reduced pressure. The resulting residue is purified by flash chromatography on silica gel, eluting with DCM/MeOH using a gradient of 100/0 to 90/10 to yield the title compound (15 g, 49% yield). ES/MS (m/z for ⁷⁹Br, ⁸¹Br): 312.0, 314.0 (M+H).

### Preparation 4

### [(3R,4R)-4-[(1R)-1-(4-bromophenyl)ethyl]-4-methyl-5-oxo-pyrrolidin-3-yl]methyl Methanesulfonate

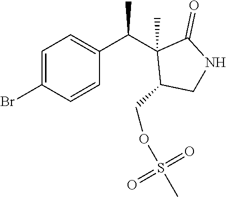 [SMILES: C[C@H](c1ccc(Br)cc1)[C@@]1(C)C(=O)NC[C@@H]1COS(C)(=O)=O]

Scheme 2, step A: To a stirred solution of (3R,4R)-3-[(1R)-1-(4-bromophenyl)ethyl]-4-(hydroxymethyl)-3-methyl-pyrrolidin-2-one (5.6 g, 18 mmol) and TEA (3.6 g, 36 mmol) in DCM (56 ml) at RT under an atmosphere of N₂ is carefully added methanesulfonyl chloride (1.7 ml, 21 mmol) dropwise. The reaction is stirred for 2 h and diluted with DCM and water. The phases are separated and the aqueous layer is extracted with more DCM. The organic extracts are combined, washed with saturated aqueous NaCl, dried over MgSO₄, filtered, and concentrated under reduced pressure to afford the title compound as a white solid (7.0 g, 96% yield). ES/MS (m/z for ⁷⁹Br, ⁸¹Br): 389.8, 391.8 (M+H).

### Preparation 5

### (3R,4R)-3-[(1R)-1-(4-bromophenyl)ethyl]-4-(fluoromethyl)-3-methyl-pyrrolidin-2-one

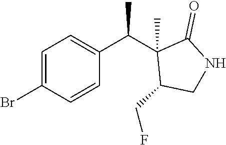 [SMILES: C[C@H](c1ccc(Br)cc1)[C@@]1(C)C(=O)NC[C@@H]1CF]

Scheme 2, step B: To a solution of [(3R,4R)-4-[(1R)-1-(4-bromophenyl)ethyl]-4-methyl-5-oxo-pyrrolidin-3-yl]methyl methanesulfonate (2.1 g, 5.2 mmol) in IPA (15 ml) is added CsF (13 g, 8.3 mmol). The mixture is heated in a microwave at 130° C. for 3 h and cooled to RT. The resulting reaction mixture is diluted with water and EtOAc. The layers are separated and the aqueous phase is extracted with EtOAc. The organic extracts are combined, dried over MgSO₄, filtered, and concentrated under reduced pressure to yield a white foam. The procedure is carried out an additional time on the same scale, and the two batches are combined and purified by flash chromatography on silica gel, eluting with cyclohexane/EtOAc, using a gradient of 70/30 to 0/100, to yield title compound (3.2 g, 88% yield) of sufficient purity for subsequent use. ES/MS (m/z for ⁷⁹Br, ⁸¹Br): 314.0, 316.0 (M+H).

### Preparation 6

### (3R,4R)-4-(fluoromethyl)-3-[(1R)-1-[4-(hydroxymethyl)phenyl]ethyl]-3-methyl-pyrrolidin-2-one

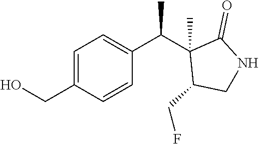 [SMILES: C[C@H](c1ccc(CO)cc1)[C@@]1(C)C(=O)NC[C@@H]1CF]

Scheme 2, step C: To a solution of (3R,4R)-3-[(1R)-1-(4-bromophenyl)ethyl]-4-(fluoromethyl)-3-methyl-pyrrolidin-2-one (3.2 g, 8.9 mmol) in toluene (80 ml) in a reaction vial is added TMEDA (1.1 g, 9.7 mmol), Pd(OAc)₂ (80 mg, 0.36 mmol) and butyldi(1-adamantyl)phosphine (0.37 g, 0.98 mmol). The vial is sealed and stirred under a 1:1 mixture of carbon monoxide and hydrogen at 95° C. and 65 psi overnight. The reaction mixture is cooled to RT, diluted with EtOAc, filtered, and concentrated under reduced pressure. The residue is purified by flash chromatography on silica gel, eluting with cyclohexane/EtOAc, using a gradient of 90/10 to 0/100, to afford 4-[(1R)-1-[(3R,4R)-4-(fluoromethyl)-3-methyl-2-oxo-pyrrolidin-3-yl]ethyl]benzaldehyde (2.6 g). This isolated material is dissolved in EtOH (104 ml), cooled to 0° C. under an atmosphere of N₂, and treated with NaBH₄ (0.45 g, 12 mmol). After 30 min, the reaction mixture is quenched by a slow addition of a saturated aqueous solution of NH₄Cl (15 mL) and water (15 mL). Once gas evolution ceases, the reaction mixture is concentrated under reduced pressure, and the resulting residue is dissolved in water and EtOAc. The layers are separated and the aqueous phase is extracted with EtOAc. The organic extracts are dried over MgSO₄, filtered, and concentrated under reduced pressure. The resulting residue is dissolved in MeOH (to a total volume of 39.2 ml), filtered, and purified by prep-HPLC (PHENOMENEX® GEMINI®-NX, 10μ, 50×150 mm C-18, 210 nm, 110 mL/min), eluting with ACN and water containing 10 mM NH₄HCO₃ and adjusted to pH˜9 with NH₄OH, using a gradient of 15% to 100% ACN over 10 min (using 4 equal injections of crude product in MeOH). The fractions containing desired material are concentrated under reduced pressure to yield the title compound as colorless oil (1.8 g, 76% yield). ES/MS (m/z): 266.0 (M+H).

### Preparation 7

### 4-(azetidin-1-yl)-2-chloro-6-methyl-pyridine

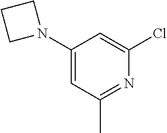

To a solution of 2,4-dichloro-6-methyl-pyridine (0.7 ml, 5.6 mmol) in toluene (10 ml) in a reaction vial is added azetidine (0.38 g, 6.7 mmol), sodium tert-butoxide (0.66 g, 6.7 mmol), 2-(di-tert-butylphosphino)biphenyl (0.19 g, 0.61 mmol) and Pd(OAc)₂ (0.14 g, 0.61 mmol). The vial is sealed, evacuated and back-filled three times with N₂, and heated with stirring at 100° C. overnight. The reaction mixture is poured into water, the layers are separated, and the aqueous phase is extracted with EtOAc. The combined organic extracts are washed with saturated aqueous NaCl, dried over MgSO₄, filtered, and concentrated under reduced pressure to afford an orange oil as a mixture of regioisomers. The regioisomers are separated by flash chromatography on silica gel, eluting with cyclohexane/EtOAc, using a gradient of 100/0 to 40/60).

The first eluting regioisomer is 2-(azetidin-1-yl)-4-chloro-6-methyl-pyridine (0.32 g, 30% yield). ¹H NMR (CDCl₃) δ 2.32-2.43 (m, 5H), 3.99-4.04 (m, 4H), 6.05 (s, 1H) and 6.45 (s, 1H). ES/MS (m/z): 183.0 (M+H).

The second eluting regioisomer is the title compound (0.48 g, 43% yield). ¹H NMR (CDCl₃) δ 2.34-2.50 (m, 5H), 3.92-3.98 (m, 4H), 5.98-6.00 (m, 1H) and 6.06-6.08 (m, 1H). ES/MS (m/z): 183.0 (M+H).

### Preparation 8

### Etherification Catalyst Mixture

A mixture of Cs₂CO₃ (80 g, 245 mmol), tris(dibenzylideneacetone)dipalladium(0) (2.3 g, 2.5 mmol), 2-(di-tert-butylphosphino)-2′,4′,6′-triisopropyl-3,6-dimethoxy-1,1′-biphenyl (5.0 g, 9.8 mmol) and toluene (500 mL) are placed in a round bottom flask. The flask is sealed, evacuated and back-filled three times with N₂, and heated at 85° C. with stirring for 1 h. The reaction mixture is cooled to RT and the solvent is removed under reduced pressure to yield a dry powder which is ground in a pestle and mortar to give 86 g of fine green powder.

### Preparation 9

### 2-chloro-4-(3-fluoroazetidin-1-yl)-6-methyl-pyridine

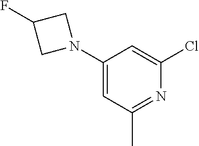

To a solution of 4-bromo-2-chloro-6-methyl-pyridine (0.97 g, 4.7 mmol) in DMSO (3.3 ml) in a reaction vial is added 3-fluoroazetidine hydrochloride (0.76 g, 6.8 mmol), L-proline (0.33 g, 2.8 mmol), Cs₂CO₃ (0.53 g, 16 mmol) and CuI (0.55 g, 2.8 mmol). The vial is sealed under an atmosphere of N₂, and heated to 95° C. with stirring overnight. The reaction mixture is diluted with EtOAc and water, the layers are separated, and the aqueous phase is extracted with EtOAc. The organic extracts are washed with saturated aqueous NaCl, dried over MgSO₄, filtered, and concentrated under reduced pressure to afford a brown oil. The resulting residue is purified by flash chromatography on silica gel, eluting with cyclohexane/EtOAc, using a gradient of 100/0 to 40/60, to obtain the title compound as a light yellow solid (422 mg, 45% yield). ES/MS (m/z): 201.0 (M+H).

### Preparation 10

### (3R,4R)-3-[(1R)-1-(4-bromophenyl)ethyl]-4-[[tert-butyl(dimethyl)silyl]oxymethyl]-3-methyl-pyrrolidin-2-one

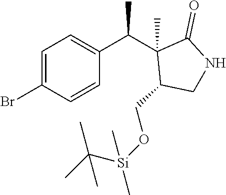 [SMILES: C[C@H](c1ccc(Br)cc1)[C@@]1(C)C(=O)NC[C@@H]1CO[Si](C)(C)C(C)(C)C]

Scheme 4, step A: To a stirred solution of (3R,4R)-3-[(1R)-1-(4-bromophenyl)ethyl]-4-(hydroxymethyl)-3-methyl-pyrrolidin-2-one (2.2 g, 7.1 mmol) in DCM (70 ml) is added tert-butyldimethylchlorosilane (1.6 g, 11 mmol), imidazole (0.72 g, 11 mmol) and 4-dimethylaminopyridine (43 mg, 0.35 mmol). The resulting mixture is stirred overnight under an atmosphere of N₂. The reaction mixture is quenched with water, the layers are separated, and the aqueous layer is extracted with DCM. The organic extracts are dried over MgSO₄, filtered, and concentrated under reduced pressure. The resulting residue is purified by flash chromatography on silica gel, eluting with cyclohexane/EtOAc, using a gradient of 100/0 to 50/50, to afford the title compound as a white solid (2.8 g, 93% yield). ES/MS (m/z for ⁷⁹Br, ⁸¹Br): 426.0, 428.0 (M+H).

### Preparation 11

### (3R,4R)-4-[[tert-butyl(dimethyl)silyl]oxymethyl]-3-[(1R)-1-[4-(hydroxymethyl)phenyl]ethyl]-3-methyl-pyrrolidin-2-one

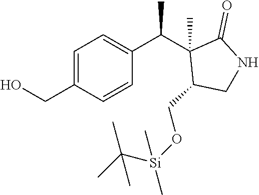 [SMILES: C[C@H](c1ccc(CO)cc1)[C@@]1(C)C(=O)NC[C@@H]1CO[Si](C)(C)C(C)(C)C]

Scheme 4, step B: To a solution of (3R,4R)-3-[(1R)-1-(4-bromophenyl)ethyl]-4-[[tert-butyl(dimethyl)silyl]oxymethyl]-3-methyl-pyrrolidin-2-one (2.8 g, 6.5 mmol) in toluene (58 ml) in a reaction vial is added TMEDA (0.82 g, 7.1 mmol), Pd(OAc)₂ (58 mg, 0.26 mmol) and butyldi(1-adamantyl)phosphine (0.27 g, 0.71 mmol). The vial is sealed and stirred under a 1:1 mixture of carbon monoxide and H₂ at 95° C. and 65 psi overnight. The reaction mixture is diluted with EtOAc, filtered, and concentrated under reduced pressure. The resulting residue is purified by flash chromatography on silica gel, eluting with cyclohexane/EtOAc, using a gradient of 100/0 to 0/100), to yield 4-[(1R)-1-[(3R,4R)-4-[[tert-butyl(dimethyl)silyl]oxymethyl]-3-methyl-2-oxo-pyrrolidin-3-yl]ethyl]benzaldehyde (2.1 g) as yellow oil of sufficient purity for subsequent use. The 4-[(1R)-1-[(3R,4R)-4-[[tert-butyl(dimethyl)silyl]oxymethyl]-3-methyl-2-oxo-pyrrolidin-3-yl]ethyl]benzaldehyde is dissolved in EtOH (64 ml), cooled to 0° C., and treated with NaBH₄ (0.28 g, 7.2 mmol). The reaction mixture is stirred under an atmosphere of nitrogen at 0° C. for 2 h and quenched by slow addition of a saturated aqueous solution of NH₄Cl (10 mL) and water (10 mL). Once gas evolution ceases, the reaction mixture is concentrated under reduced pressure. The resulting residue is dissolved in water and EtOAc. The layers are separated and the aqueous phase is extracted with EtOAc. The organic extracts are dried over MgSO₄, filtered, and concentrated under reduced pressure. The resulting residue is purified by flash chromatography on silica gel, eluting with cyclohexane/EtOAc, using a gradient of 100/0 to 100/0, to yield the title compound (1.5 g) as a colorless oil of sufficient purity for subsequent use. ES/MS (m/z): 378.2 (M+H).

### Preparation 12

### (3R,4R)-3-[(1R)-1-[4-[[4-(azetidin-1-yl)-6-methyl-2-pyridyl]oxymethyl]phenyl]ethyl]-4-[[tert-butyl(dimethyl)silyl]oxymethyl]-3-methyl-pyrrolidin-2-one

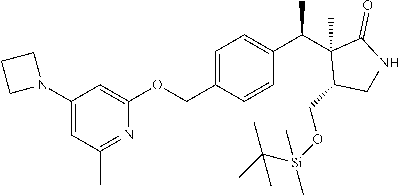 [SMILES: Cc1cc(N2CCC2)cc(OCc2ccc([C@@H](C)[C@@]3(C)C(=O)NC[C@@H]3CO[Si](C)(C)C(C)(C)C)cc2)n1]

Scheme 4, step C: To a solution of (3R,4R)-4-[[tert-butyl(dimethyl)silyl]oxymethyl]-3-[(1R)-1-[4-(hydroxymethyl)phenyl]ethyl]-3-methyl-pyrrolidin-2-one (0.22 g, 0.54 mmol) in toluene (5.4 ml) in a reaction vial is added 4-(azetidin-1-yl)-2-chloro-6-methyl-pyridine (0.13 g, 0.64 mmol) and Etherification Catalyst Mixture (0.60 g). The vial is sealed, evacuated and back-filled three times with N₂, and heated to 85° C. with stirring for 4 h. The reaction mixture is cooled to RT and treated with more 4-(azetidin-1-yl)-2-chloro-6-methyl-pyridine (37 mg, 0.19 mmol) and Etherification Catalyst Mixture (0.30 g). The vial is sealed, evacuated and back-filled three times with N₂, and heated to 85° C. with stirring for 13 h. The reaction mixture is poured onto a saturated aqueous solution of NH₄Cl. The layers are separated and the aqueous phase is extracted with DCM. The organic extracts are dried over MgSO₄, filtered, and concentrated under reduced pressure to afford an orange oil. The crude product is purified by flash chromatography on silica gel, eluting with cyclohexane/EtOAc, using a gradient of 100/0 to 0/100, to yield the title compound as a yellow oil (0.24 g, 85% yield). ES/MS (m/z): 524.2 (M+H).

### Preparation 13

### (3R,4R)-3-[(1R)-1-[4-[[4-(azetidin-1-yl)-6-methyl-2-pyridyl]oxymethyl]phenyl]ethyl]-4-(hydroxymethyl)-3-methyl-pyrrolidin-2-one

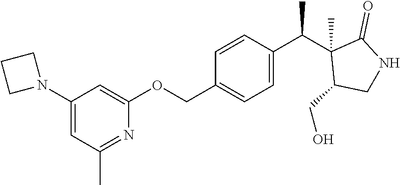 [SMILES: Cc1cc(N2CCC2)cc(OCc2ccc([C@@H](C)[C@@]3(C)C(=O)NC[C@@H]3CO)cc2)n1]

Scheme 4, step D: To a solution of (3R,4R)-3-[(1R)-1-[4-[[4-(azetidin-1-yl)-6-methyl-2-pyridyl]oxymethyl]phenyl]ethyl]-4-[[tert-butyl(dimethyl)silyl]oxymethyl]-3-methyl-pyrrolidin-2-one (0.24 g, 90% pure by LCMS UV, 0.41 mmol) in THF (4.1 ml) under an atmosphere of N₂ is added a 1 N solution of tetrabutylammonium fluoride in THF (0.49 ml, 0.49 mmol). The reaction mixture is stirred at RT for 2.5 h and treated with additional 1 N solution of tetrabutylammonium fluoride in THF (0.21 ml, 0.21 mmol). The resulting mixture is stirred at RT overnight and quenched with the addition of saturated aqueous solution of NH₄Cl. The layers are separated and the aqueous phase is extracted with EtOAc. The organic extracts are dried over MgSO₄, filtered, and concentrated under reduced pressure. The resulting residue is dissolved in MeOH (to a total volume of 9.8 ml), filtered, and purified by prep-HPLC (PHENOMENEX® GEMINI®-NX, 10μ, 50×150 mm C-18, 219 nm, 120 mL/min), eluting with ACN and water adjusted to pH˜9 with conc. aqueous NH₄OH solution [0.5 ml of conc. NH₄OH per 2.5 L of water], using a gradient of 15% to 100% ACN over 11 minutes, to afford the title compound (99 mg, 59% yield). ES/MS (m/z): 410.0 (M+H).

### Preparation 14

### [(3R,4R)-4-[(1R)-1-[4-[[4-(azetidin-1-yl)-6-methyl-2-pyridyl]oxymethyl]phenyl]ethyl]-4-methyl-5-oxo-pyrrolidin-3-yl]methyl 4-methylbenzenesulfonate

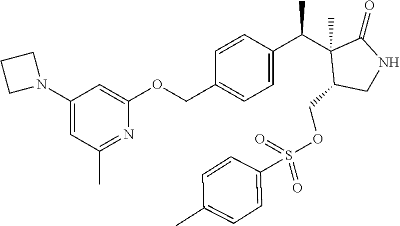 [SMILES: Cc1ccc(S(=O)(=O)OC[C@H]2CNC(=O)[C@]2(C)[C@H](C)c2ccc(COc3cc(N4CCC4)cc(C)n3)cc2)cc1]

Scheme 4, step E: To an ice bath chilled solution of (3R,4R)-3-[(1R)-1-[4-[[4-(azetidin-1-yl)-6-methyl-2-pyridyl]oxymethyl]phenyl]ethyl]-4-(hydroxymethyl)-3-methyl-pyrrolidin-2-one (50 mg, 0.12 mmol) and TEA (62 mg, 0.61 mmol) in DCM (5 ml) under N₂ is added p-toluenesulfonyl chloride (58 mg, 0.31 mmol). The mixture is stirred to RT overnight. The reaction mixture is treated with additional TEA (62 mg, 0.61 mmol) and p-toluenesulfonyl chloride (58 mg, 0.31 mmol) and stirred at RT for 7 h. The mixture is treated with additional TEA (62 mg, 0.61 mmol) and p-toluenesulfonyl chloride (58 mg, 0.31 mmol) and stirred at RT overnight. The reaction mixture is concentrated under reduced pressure and purified by flash chromatography on silica gel, eluting with cyclohexane/EtOAc, using a gradient of 90/10 to 0/100, to yield the title compound as a colorless glass (35 mg, 47% yield). ES/MS (m/z): 564.2 (M+H).

### Preparation 15

### (3R,4R)-3-[(1R)-1-(4-bromophenyl)ethyl]-3,4-dimethylpyrrolidine-2,5-dione

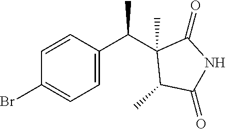 [SMILES: C[C@H](c1ccc(Br)cc1)[C@@]1(C)C(=O)NC(=O)[C@@H]1C]

Scheme 3, step A: To a solution (35)-3-[(1R)-1-(4-bromophenyl)ethyl]-3-methylpyrrolidine-2,5-dione (1.2 g, 4.1 mml) in anhydrous THF (34 mL) under a nitrogen atmosphere immersed in a water bath at RT, is added 0.9 M lithium bis(trimethylsilyl)amide in THF (10 mL, 9.3 mmol) portion-wise. The reaction mixture is stirred for 1.5 hr. A solution of methyl iodide (previously passed through basic alumina) in THF (2 mL) is added drop wise over 5 min. After stirring for 1 hr the reaction is quenched using saturated aqueous NH₄Cl. The reaction is concentrated to remove most of the THF, and the residue is partitioned between EtOAc and water. The aqueous layer is removed and the organic layer is dried over Na₂SO₄, filtered, and concentrated under reduced pressure. The resulting residue is purified using normal phase chromatography on silica (80 g), eluting with a gradient of 20-30% EtOAc/hexanes over 30 min, to obtain the title compound as colorless oil (1.2 g, 95% yield) after solvent evaporation. ¹H NMR (400 MHz, CDCl₃): δ 0.94 (d, J=7.5 Hz, 3H), 1.16 (s, 3H), 1.37 (d, J=7.1 Hz, 3H), 3.03 (q, J=7.5 Hz, 1H), 3.16 (q, J=7.1 Hz, 1H), 7.08-7.11 (d, 2H), 7.45-7.49 (d, 2H), 7.90 (bs, 1H). ES/MS (m/z for ⁷⁹Br, ⁸¹Br): 309.8, 311.8 (M+H).

### Preparation 16

### (3R,4R)-3-[(1R)-1-(4-bromophenyl)ethyl]-3,4-dimethyl-pyrrolidin-2-one

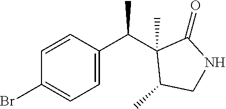 [SMILES: C[C@H](c1ccc(Br)cc1)[C@@]1(C)C(=O)NC[C@@H]1C]

Scheme 3, step B: To a solution of (3R,4R)-3-[(1R)-1-(4-bromophenyl)ethyl]-3,4-dimethyl-pyrrolidine-2,5-dione (3.86 g, 12.2 mmol) in THF (50 ml) stirring under nitrogen and cooled in an ice/water bath is added borane dimethyl sulfide complex (6.10 ml, 61.1 mmol) portion wise over 25 minutes. The mixture is stirred at RT for 21 h. The reaction mixture is cooled in an ice/water bath and carefully quenched with MeOH (10 ml). The reaction is concentrated under reduced pressure, dissolved in MeOH (25 ml) and concentrated. The concentrate is dissolved in TFA (20 ml), cooled in an ice/water bath, and NaBH₄ (2.36 g, 61.1 mmol) is added portion wise over 30 min, while purging the reaction flask with N₂. The reaction mixture is stirred an additional 30 min before quenching with ice/water (100 ml) and extracting with EtOAc (3×50 ml). The combined organic extracts are washed with saturated aqueous NaCl, dried over Na₂SO₄, filtered, and concentrated under reduced pressure. The crude product is purified by flash chromatography on silica gel, eluting with hexane/EtOAc, using a gradient of 95/5 to 25/75, to yield the title compound as a white solid (1.84 g, 51% yield). ES/MS (m/z for ⁷⁹Br, ⁸¹Br): 295.9/297.9 (M+H).

### Preparation 17

### 4-[(1R)-1-[(3R,4R)-3,4-dimethyl-2-oxo-pyrrolidin-3-yl]ethyl]benzaldehyde

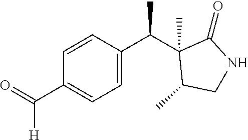 [SMILES: C[C@H](c1ccc(C=O)cc1)[C@@]1(C)C(=O)NC[C@@H]1C]

Scheme 3, step C: To a 100 ml Parr autoclave is charged (3R,4R)-3-[(1R)-1-(4-bromophenyl)ethyl]-3,4-dimethyl-pyrrolidin-2-one (1.75 g, 5.91 mmol), palladium(II) acetate (54 mg, 0.24 mmol), butyldi-1-adamantylphosphine (CataCXium® A, 255 mg, 0.675 mmol), anhydrous toluene (50 ml) and TMEDA (1.0 ml, 6.6 mmol). The autoclave is sealed, the reaction mixture is placed under an atmosphere of synthesis gas (H₂/CO (1:1, 75 psi), heated to 95° C., and stirred for 16 h. The reaction mixture is cooled and the suspension is filtered over a pad of diatomaceous earth. The filter cake is washed with EtOAc, and the collected filtrates are concentrated under reduced pressure to afford amber oil. The crude product is purified by flash chromatography on silica gel, eluting with DCM/MeOH, using a gradient from 100:0 to 9:1, to give the title compound (1.36 g, 83% yield). ES/MS (m/z): 246.0 (M+H).

### Preparation 18

### (3R,4R)-3-[(1R)-1-[4-(hydroxymethyl)phenyl]ethyl]-3,4-dimethyl-pyrrolidin-2-one

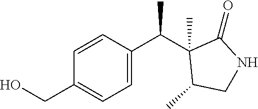 [SMILES: C[C@H](c1ccc(CO)cc1)[C@@]1(C)C(=O)NC[C@@H]1C]

Scheme 3, step D: In a single portion, NaBH₄ (263 mg, 6.82 mmol) is added to a suspension of 4-[(1R)-1-[(3R,4R)-3,4-dimethyl-2-oxo-pyrrolidin-3-yl]ethyl]benzaldehyde (1.36 g, 4.55 mmol) in EtOH (60 ml), cooled in an ice/water bath. After 45 min, the reaction is quenched with water (10 ml) and concentrated under reduced pressure. The resulting concentrate is diluted with water (50 ml) and extracted with EtOAc (2×50 ml). The combined organic extracts are washed with saturated aqueous NaCl, dried over Na₂SO₄, filtered, and concentrated under reduced pressure. The crude product is purified by flash chromatography on silica gel, eluting with DCM/MeOH, using a gradient from 100:0 to 9:1, to give the title compound (1.02 g, 91% yield). ES/MS (m/z): 248.8 (M+H).

### Preparation 19

### tert-butyl-[1-(2-chloro-6-methyl-4-pyridyl)azetidin-3-yl]oxy-dimethyl-silane

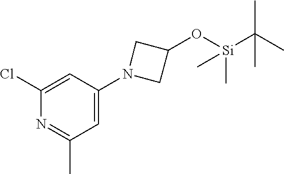 [SMILES: Cc1cc(N2CC(O[Si](C)(C)C(C)(C)C)C2)cc(Cl)n1]

A reaction vessel is charged with 2,4-dichloro-6-methylpyridine (2.05 g, 12.3 mmol), 3-hydroxyazetidine hydrochloride (2.06 g, 18.4 mmol), Cs₂CO₃ (14.0 g, 43.0 mmol), L-proline (856 mg, 7.3 mmol), CuI (1.4 g, 7.3 mmol) and DMSO (15 ml). The vessel is sealed with a septum, evacuated, and back-filled four times with N₂. The reaction mixture is heated at 90° C. for 16 h and cooled to RT. The reaction mixture is filtered through paper, and the filter cake is washed with EtOAc. The filtrate is diluted with water and extracted with EtOAc (3×50 ml). The combined organic extracts are washed with saturated aqueous NaCl, dried over Na₂SO₄, filtered, and concentrated to give a crude mixture of regioisomers (2.22 g).

The crude mixture of regioisomers is partially dissolved in DCM (50 ml) and treated with t-butyl dimethylsilyl chloride (2.60 g, 16.8 mmol) and imidazole (1.15 g, 16.8 mmol). After stirring at RT for 45 min, the reaction is diluted with water (75 ml) and extracted with DCM (2×50 ml). The combined organic extracts are dried over Na₂SO₄, filtered, and concentrated in vacuo. The crude product is purified by flash chromatography on silica gel, eluting with EtOAc/hexane, using a gradient from 2:98 to 30:70, to obtain the title compound (1.88 g, 54% yield) as the second eluting regioisomer. ES/MS (m/z for ³⁵Cl, ³⁷Cl): 313.0/315.0 (M+H).

### Preparation 20

### 1-(2-chloro-6-methyl-4-pyridyl)azetidin-3-ol

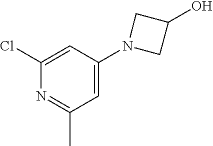

A solution of tert-butyl-[1-(2-chloro-6-methyl-4-pyridyl)azetidin-3-yl]oxy-dimethyl-silane (3.03 g, 9.67 mmol) in THF (50 ml) is cooled in an ice/water bath and treated with a 1.0 M solution of TBAF in THF (39 ml, 39 mmol). After stirring at RT for 1 h, the reaction mixture is concentrated under reduced pressure. The concentrate is dissolved in DCM (150 ml), washed sequentially with water and saturated aqueous NaCl, dried over Na₂SO₄, filtered, and concentrated under reduced pressure. The crude product is purified by flash chromatography on silica gel, eluting with EtOAc/hexane, using a gradient from 10:90 to 100:0, to obtain the title compound (1.69 g, 88% yield). ES/MS (m/z for ³⁵Cl, ³⁷Cl): 199.0/201.0 (M+H).

### Preparation 21

### (3R,4R)-3-[(1R)-1-[4-[[4-(3-hydroxyazetidin-1-yl)-6-methyl-2-pyridyl]oxymethyl]phenyl]ethyl]-3,4-dimethyl-pyrrolidin-2-one

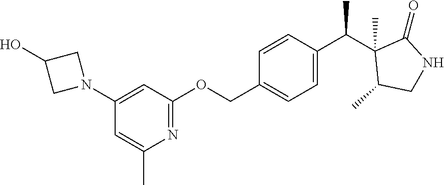 [SMILES: Cc1cc(N2CC(O)C2)cc(OCc2ccc([C@@H](C)[C@@]3(C)C(=O)NC[C@@H]3C)cc2)n1]

Scheme 5, step A: A reaction vessel is charged with (3R,4R)-3-[(1R)-1-[4-(hydroxymethyl)phenyl]ethyl]-3,4-dimethyl-pyrrolidin-2-one (1.0 g, 4.1 mmol), 1-(2-chloro-6-methyl-4-pyridyl)azetidin-3-ol (1.7 mg, 8.5 mmol), Cs₂CO₃ (3.5 g, 10.8 mmol), tBuBrettPhos (216 mg, 0.445 mmol), Pd₂(dba)₃ (102 mg, 0.11 mmol) and toluene (30 ml). The vessel is sealed with a septum, evacuated, and back-filled four times with N₂. The reaction mixture is heated at 100° C. for 14 h and cooled to RT. The reaction mixture is poured into saturated aqueous NH₄Cl (40 ml) and extracted with DCM (3×30 ml). The combined organic extracts are dried over Na₂SO₄, filtered, and concentrated under reduced pressure. The crude product is purified by flash chromatography on silica gel, eluting with MeOH/DCM, using a gradient from 0:100 to 10:90. The product is further purified by high pH reverse phase chromatography on C18 silica, eluting with ACN/10 mM aqueous NH₄HCO₃, using a gradient from 25:75 to 42:58, to obtain the title compound (412 mg, 22% yield). ES/MS (m/z): 410.2 (M+H).

### Preparation 22

### [1-[2-[[4-[(1R)-1-[(3R,4R)-3,4-dimethyl-2-oxo-pyrrolidin-3-yl]ethyl]phenyl]methoxy]-6-methyl-4-pyridyl]azetidin-3-yl] 4-methylbenzenesulfonate

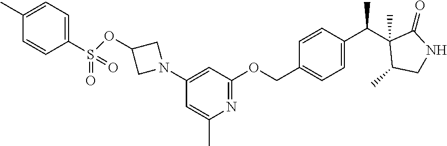 [SMILES: Cc1ccc(S(=O)(=O)OC2CN(c3cc(C)nc(OCc4ccc([C@@H](C)[C@@]5(C)C(=O)NC[C@@H]5C)cc4)c3)C2)cc1]

Scheme 5, step B: To a solution of (3R,4R)-3-[(1R)-1-[4-[[4-(3-hydroxyazetidin-1-yl)-6-methyl-2-pyridyl]oxymethyl]phenyl]ethyl]-3,4-dimethyl-pyrrolidin-2-one (115 mg, 0.26 mmol) and TEA (131 mg, 1.28 mmol) in DCM (5 ml) is added p-toluenesulfonyl chloride (122 mg, 0.64 mmol) under nitrogen. The reaction mixture is stirred at RT for 17 h. The reaction is concentrated under reduced pressure and the resulting residue is purified by flash chromatography on silica gel, eluting with hexane/EtOAc, using a gradient of 90/10 to 0/100), to obtain the title compound as a white foam (114 mg, 79% yield). ES/MS (m/z): 564.2 (M+H).

### Preparation 23

### (3R,4R)-4-(fluoromethyl)-3-[(1R)-1-[4-[[4-(3-hydroxyazetidin-1-yl)-6-methyl-2-pyridyl]oxymethyl]phenyl]ethyl]-3-methyl-pyrrolidin-2-one

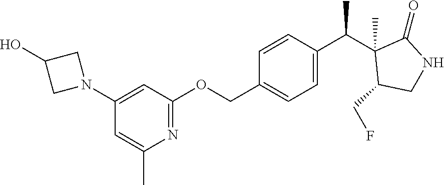 [SMILES: Cc1cc(N2CC(O)C2)cc(OCc2ccc([C@@H](C)[C@@]3(C)C(=O)NC[C@@H]3CF)cc2)n1]

Scheme 2, step D: To a solution of (3R,4R)-4-(fluoromethyl)-3-[(1R)-1-[4-(hydroxymethyl)phenyl]ethyl]-3-methyl-pyrrolidin-2-one (151.3 mg, 0.56 mmol) in toluene (5.6 mL) is added 1-(2-chloro-6-methyl-4-pyridyl)azetidin-3-ol (224.3 mg, 1.13 mmol,) and CatKit Etherification mix (630.1 mg, 1.69 mmol). The vial is sealed and the reaction mixture is evacuated and back-filled with nitrogen 3 times. The resulting mixture is stirred at 85° C. for 20 h. After cooling to RT, the reaction mixture is poured into a saturated aqueous solution of NH₄Cl. The layers are separated and the aqueous phase extracted with DCM. The combined organic extracts are dried over MgSO₄, filtered, and concentrated under reduced pressure to afford orange oil. The residue is dissolved in MeOH and purified by prep-HPLC (C-18 PHENOMENEX® Gemini-NX, 10μ, 50×150 mm, 120 mL/min, 11 min run, 219 nm), eluting with a gradient of 15%-100% ACN in a solution of water adjusted to approx. pH˜9 with conc. NH₄OH (0.5 ml of conc. NH₄OH per 2.5 L of water). The solvent is evaporated from the desired product fractions, and the resulting residue is further purified by preparative SFC (BzS column, 150×4.6 mm, 5μ 120 g/min, outlet pressure 100.0 bar). eluting with 17% of a mixture of MeOH containing 0.2% DMEA in CO₂, to give the title compound (27 mg, 11% yield) after solvent evaporation. ES/MS (m/z): 428.2 (M+H).

### Preparation 24

### (3R,4R)-4-[[tert-butyl(dimethyl)silyl]oxymethyl]-3-[(1R)-1-[4-[[4-(3-fluoroazetidin yl)-6-methyl-2-pyridyl]oxymethyl]phenyl]ethyl]-3-methyl-pyrrolidin-2-one

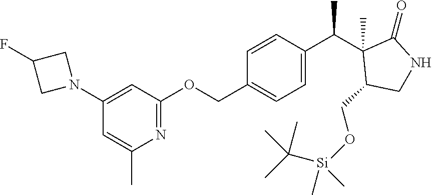 [SMILES: Cc1cc(N2CC(F)C2)cc(OCc2ccc([C@@H](C)[C@@]3(C)C(=O)NC[C@@H]3CO[Si](C)(C)C(C)(C)C)cc2)n1]

To a solution of (3R,4R)-4-[[tert-butyl(dimethyl)silyl]oxymethyl]-3-[(1R)-1-[4-(hydroxymethyl)phenyl]ethyl]-3-methyl-pyrrolidin-2-one (462.3 mg, 1.13 mmol) in toluene (11.26 mL) is added 2-chloro-4-(3-fluoroazetidin-1-yl)-6-methyl-pyridine (248.6 mg, 1.24 mmol) and CatKit Etherification mix (1257 mg, 3.38 mmol). The vial is sealed and the reaction mixture is evacuated and back-filled with N₂ 3 times, and stirred at 85° C. overnight. The reaction mixture is poured into a saturated aqueous solution of NH₄Cl. The layers are separated and the aqueous phase extracted with DCM. The combined organic extracts are dried over MgSO₄, filtered, and concentrated under reduced pressure to afford orange oil. The resulting residue is purified by flash chromatography over silica gel, eluting with a gradient of 0-100% EtOAc in cyclohexane, to obtain the title compound (318 mg, 52% yield) as a yellow foam, after evaporation of the desired chromatographic fractions. ES/MS (m/z): 428.2 (M+H).

### Preparation 25

### (3R,4R)-3-[(1R)-1-[4-[[4-(3-fluoroazetidin-1-yl)-6-methyl-2-pyridyl]oxymethyl]phenyl]ethyl]-4-(hydroxymethyl)-3-methyl-pyrrolidin-2-one

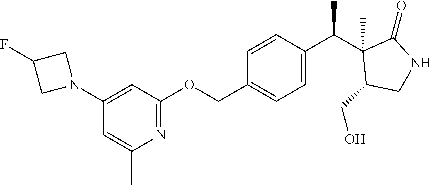 [SMILES: Cc1cc(N2CC(F)C2)cc(OCc2ccc([C@@H](C)[C@@]3(C)C(=O)NC[C@@H]3CO)cc2)n1]

To a solution of (3R,4R)-4-[[tert-butyl(dimethyl)silyl]oxymethyl]-3-[(1R)-1-[4-[[4-(3-fluoroazetidin-1-yl)-6-methyl-2-pyridyl]oxymethyl]phenyl]ethyl]-3-methyl-pyrrolidin-2-one (370.7 mg, 0.59 mmol) in THF (5.9 mL,) under N₂ is added dropwise a 1M solution of TBAF in THF (1.0 mL), and the reaction mixture is stirred at RT for 6 h. A saturated aqueous solution of NH₄Cl is added and the layers are separated. The aqueous phase extracted with EtOAc, dried over MgSO₄, filtered, and concentrated under reduced pressure. The resulting residue is dissolved in MeOH (9.8 ml), filtered, and the methanolic filtrate is purified by preparative HPLC (C-18 PHENOMENEX® Gemini-NX, 10μ, 50×150 mm, 120 mL/min, 11 min run, 219 nm), eluting with a gradient of 15%-100% ACN in a solution of water adjusted to approx. pH˜9 with conc. NH₄OH (0.5 ml of conc. NH₄OH per 2.5 L of water) to afford the title compound (180 mg, 72% yield) as a yellow oil, after evaporation of the chromatographic fractions. ES/MS (m/z): 428.2 (M+H).

### Example 1

### (3R,4R)-3-[(1R)-1-[4-[[4-(azetidin-1-yl)-6-methyl-2-pyridyl]oxymethyl]phenyl]ethyl]-4-(fluoromethyl)-3-methyl-pyrrolidin-2-one

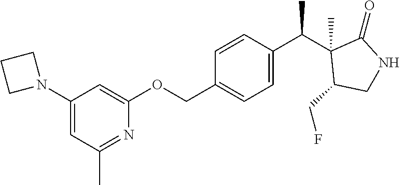 [SMILES: Cc1cc(N2CCC2)cc(OCc2ccc([C@@H](C)[C@@]3(C)C(=O)NC[C@@H]3CF)cc2)n1]

Scheme 2, step D: To a solution of (3R,4R)-4-(fluoromethyl)-3-[(1R)-1-[4-(hydroxymethyl)phenyl]ethyl]-3-methyl-pyrrolidin-2-one (0.25 g, 0.92 mmol) in toluene (9 ml) in a reaction vial is added 4-(azetidin-1-yl)-2-chloro-6-methyl-pyridine (0.22 g, 1.1 mmol) and Etherification Catalyst Mixture (1.0 g). The vial is sealed, evacuated and back-filled three times with N₂, and heated to 85° C. with stirring for 20 h. The reaction mixture is poured onto a saturated aqueous solution of NH₄Cl. The layers are separated and the aqueous phase is extracted with DCM. The organic extracts are dried over MgSO₄, filtered, and concentrated under reduced pressure to afford orange oil. The oil is dissolved in MeOH (to a total volume of 9.8 ml), filtered, and purified by prep-HPLC (PHENOMENEX® GEMINI®-NX, 10μ, 50×150 mm C-18, 219 nm, 120 mL/min), eluting with ACN and water adjusted to pH˜9 with conc. aqueous NH₄OH solution [0.5 ml of conc. NH₄OH per 2.5 L of water], using a gradient of 15% to 100% ACN over 11 min, to afford the title compound (0.21 g, 54% yield). ES/MS (m/z): 412.2 (M+H).

### Example 2

### (3R,4R)-3-[(1R)-1-[4-[[4-(3-fluoroazetidin-1-yl)-6-methyl-2-pyridyl]oxymethyl]phenyl]ethyl]-3,4-dimethyl-pyrrolidin-2-one

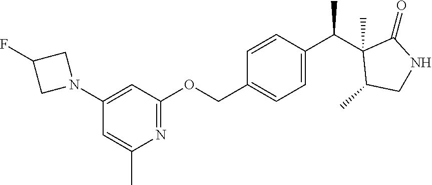 [SMILES: Cc1cc(N2CC(F)C2)cc(OCc2ccc([C@@H](C)[C@@]3(C)C(=O)NC[C@@H]3C)cc2)n1]

Scheme 3, step E: A reaction vessel is charged with (3R,4R)-3-[(1R)-1-[4-(hydroxymethyl)phenyl]ethyl]-3,4-dimethyl-pyrrolidin-2-one (101 mg, 0.401 mmol), 2-chloro-4-(3-fluoroazetidin-1-yl)-6-methyl-pyridine (123 mg, 0.61 mmol), Cs₂CO₃ (292 mg, 0.90 mmol), tBuBrettPhos (18 mg, 0.037 mmol), Pd₂(dba)₃ (8 mg, 0.009 mmol) and toluene (6 ml). The vessel is sealed with a septum, evacuated, and back-filled four times with N₂. The reaction mixture is heated at 100° C. for 18 h and cooled to RT. The reaction mixture is poured into saturated aqueous NH₄Cl (20 ml) and extracted with EtOAc (2×25 ml). The combined organic layers are dried over Na₂SO₄, filtered, and concentrated. The crude product is purified by flash chromatography on silica gel, eluting with EtOAc/hexane, using a gradient from 1:9 to 100:0, to obtain the title compound (123 mg, 71% yield). ES/MS (m/z): 412.2 (M+H).

### Example 3

### (3R,4R)-3-[(1R)-1-[4-[[4-(3-fluoroazetidin-1-yl)-6-methyl-2-pyridyl]oxymethyl]phenyl]ethyl]-4-(fluoromethyl)-3-methyl-pyrrolidin-2-one

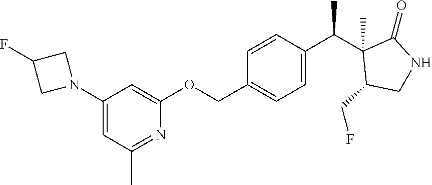 [SMILES: Cc1cc(N2CC(F)C2)cc(OCc2ccc([C@@H](C)[C@@]3(C)C(=O)NC[C@@H]3CF)cc2)n1]

Scheme 2, step D: To a solution of (3R,4R)-4-(fluoromethyl)-3-[(1R)-1-[4-(hydroxymethyl)phenyl]ethyl]-3-methyl-pyrrolidin-2-one (0.15 g, 0.55 mmol) in toluene (5.5 ml) in a reaction vial is added 2-chloro-4-(3-fluoroazetidin-1-yl)-6-methyl-pyridine (0.13 g, 0.66 mmol) and Etherification Catalyst Mixture (0.61 g). The vial is sealed, evacuated and back-filled three times with N₂, and heated to 85° C. with stirring for 16 h. The reaction mixture is poured onto a saturated aqueous solution of NH₄Cl. The layers are separated and the aqueous phase is extracted with DCM. The organic extracts are dried over MgSO₄, filtered, and concentrated under reduced pressure to orange oil. The resulting residue is dissolved in MeOH (to a total volume of 9.8 ml), filtered, and purified by prep-HPLC (PHENOMENEX® GEMINI®-NX, 10μ, 50×150 mm C-18, 219 nm, 120 mL/min), eluting with ACN and water adjusted to pH˜9 with conc. aqueous NH₄OH [0.5 ml of conc. NH₄OH per 2.5 L of water], using a gradient of 15% to 100% ACN over 11, to afford the title compound as a brown foam (0.14 g, 58% yield). ES/MS (m/z): 430.2 (M+H).

### General Preparation for [¹⁸F]Fluoride Reagent

The synthesis of [¹⁸F]labelled compounds are performed using a GE TRACERlab® FX_{F-N} automated radiosynthesizer module with a starting activity of 1-2 Ci. Typical synthesis time is ˜60±5 min and the range of decay corrected yield is 18-35%. [¹⁸F]fluoride in a shipping vial (obtained from a cyclotron facility) is transferred onto and trapped on an ion exchange cartridge. The [¹⁸F]fluoride is eluted with a solution of K₂CO₃ and Kryptofix 222 into the reaction vessel of the module. The solution is first evaporated by heating at 95° C. for 4 min under vacuum and helium flow. ACN (1 mL) is added to the vial and the evaporation is continued under the same conditions for 2 min. After a second addition of ACN (1 mL), final evaporation is carried out at 95° C. for 2 min under vacuum and helium flow to afford anhydrous Kryptofix 222-K₂CO₃[¹⁸F]fluoride.

### Example 4

### (3R,4R)-3-[(1R)-1-[4-[[4-(3-[¹⁸F]fluoroazetidin-1-yl)-6-methyl-2-pyridyl]oxymethyl]phenyl]ethyl]-3,4-dimethyl-pyrrolidin-2-one

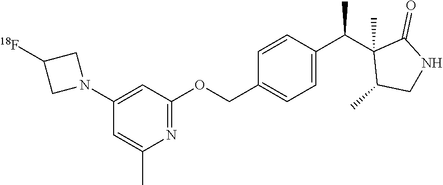 [SMILES: Cc1cc(N2CC([18F])C2)cc(OCc2ccc([C@@H](C)[C@@]3(C)C(=O)NC[C@@H]3C)cc2)n1]

Scheme 5, step C: To a reaction vial containing anhydrous Kryptofix 222-K₂CO₃[¹⁸F]fluoride at 60° C. under anhydrous He/air is added a solution of [1-[2-[[4-[(1R)-1-[(3R,4R)-3,4-dimethyl-2-oxo-pyrrolidin-3-yl]ethyl]phenyl]methoxy]-6-methyl-4-pyridyl]azetidin-3-yl] 4-methylbenzenesulfonate (1 mg) in anhydrous DMSO (1 mL). The reaction mixture is heated at 120° C. for 10 min and the reactor is cooled to 40° C., diluted with ACN/WFI, and loaded a semi-preparative HPLC under anhydrous He/air within the GE TRACERlab® FX_{F-N} module (PHENOMENEX® LUNA® C18(2) column, 10 μm, 250×10 mm; Waters XBRIDGE′ column, 5 μm, 250×10 mm; or Agilent ZORBAX® Eclipse column, 5 μm, 250×10 mm), eluting with a 60/40 (v/v) mixture ACN/5 mM aqueous NH₄OAc at 4 mL/min. The product fraction is collected in a flask containing ascorbic acid (10 mg/mL) in WFI (20 mL). The diluted product mixture is passed through a tC18 solid-phase extraction cartridge and the cartridge is rinsed with 10 mL of ascorbic acid (10 mg/mL) in WFI. The radiolabeled product is eluted from the SPE cartridge with 200-proof USP grade EtOH (1 mL) into a formulation flask, pre-loaded with 10 mL of formulation base (ascorbic acid in 0.9 M aqueous NaCl). The cartridge is rinsed with 4 mL of formulation base and the rinse is mixed with the contents of the formulation flask. The resulting solution is passed through a sterilizing 0.2 μm membrane filter into a sterile, filter-vented vial pre-filled with 15 mL of 0.9 M aqueous NaCl. A single preparation is used during this synthesis with a decay corrected yield of 30.9%.

### Example 5

### (3R,4R)-3-[(1R)-1-[4-[[4-(azetidin-1-yl)-6-methyl-2-pyridyl]oxymethyl]phenyl]ethyl]-4-([¹⁸F]fluoromethyl)-3-methyl-pyrrolidin-2-one

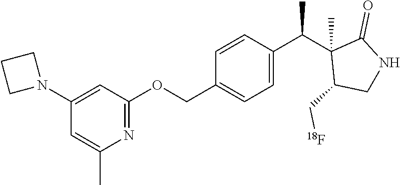 [SMILES: Cc1cc(N2CCC2)cc(OCc2ccc([C@@H](C)[C@@]3(C)C(=O)NC[C@@H]3C[18F])cc2)n1]

Scheme 4, step F: The compound of Example 5 may be prepared under conditions analogous to those described in Example 4, using anhydrous Kryptofix 222-K₂CO₃[¹⁸F]fluoride and [(3R,4R)-4-[(1R)-1-[4-[[4-(azetidin-1-yl)-6-methyl-2-pyridyl]oxymethyl]phenyl]ethyl]-4-methyl-5-oxo-pyrrolidin-3-yl]methyl 4-methylbenzenesulfonate (1 mg). A total of 9 preparations are used during this synthesis with an average decay corrected yield of 19.8%±7.9%.

### Inhibition of cAMP Production by CGRP Receptor Antagonists

The hCGRP (human calcitonin gene-related peptide) receptor is functionally coupled to the Gas proteins. Stimulation by hCGRP results in an increased synthesis of intracellular cAMP and can be blocked by the addition of receptor antagonists. Receptor activity is thus a reflection of the amount of cAMP present within cells which can be detected using standard in vitro technology.

Cell Culture: Cultured SK-N-MC neuroblastoma cells that endogenously express the hCGRP receptor (ATCC) are grown in Eagle's Minimum essential medium (HYCLONE™) supplemented with 10% heat-inactivated Fetal bovine serum (FBS; GIBCO®), Non-Essential Amino Acids (GIBCO®), 1 mM sodium pyruvate, 2 mM L-glutamine, 100 U/mL of penicillin, and 10 μg/mL of streptomycin to about 70% confluency. After providing fresh medium, the cells are incubated at 37° C. overnight. On the day of the assay, cells are detached using ACCUTASE® (MP Biomedicals), resuspended in assay buffer [Hank's Balanced Salt Solution/Dulbecco's phosphate-buffered saline with 100 mg/mL each of CaCl₂ and MgCl₂ mixed 1:2, 3.3 mM 4-(2-hydroxyethyl)-1-piperazineethanesulfonic acid, 0.03% bovine serum albumin, and 0.5 mM 1-methyl-3-isobutylxanthine (as inhibitor of cAMP)], and seeded 3-5K/well into 384-well, poly-D-lysine coated white plates (BD Biosciences).

Inhibition of cAMP Production: For dose-response studies, compounds are serially diluted 1:3 in dimethyl sulfoxide and then 1:10 into assay buffer. Human CGRP (0.8 nM; Bachem) as a receptor-specific agonist for the hCGRP receptor is mixed with diluted compound and added to the cells as the challenge stimulant at their EC₈₀ concentrations.

Data Analysis: The amount of intracellular cAMP is quantitated using HTRF technology (Cisbio) as per vendor instructions. Briefly, cAMP-d2 conjugate and anti-cAMP-cryptate conjugate in lysis buffer are incubated with the treated cells at RT for 90 min. The HTRF signal is immediately detected using an ENVISION® plate reader (Perkin-Elmer) to calculate the ratio of fluorescence at 665 to 620 nM. The raw data are converted to cAMP amount (pmole/well) using a cAMP standard curve generated for each experiment. Relative EC₅₀ values are calculated from the top-bottom range of the concentration response curve using a four-parameter logistic curve fitting program (ACTIVITYBASE® v5.3.1.22 or GENEDATA SCREENER® v12.0.4), and K_{b} values are estimated as agonist-corrected IC₅₀ values using the equation:

K_{b}=(IC₅₀)/[1+([Agonist]/EC₅₀)].

Estimated K_{b} values are reported as mean values±SEM, averaged from the number of runs (n).

Following the procedure essentially as described above, compound of Examples 1-3 have K_{b} measured at human CGRP as shown in Table 1. These data demonstrate that the compounds of Examples 1-3 are antagonists of the human CGRP receptor in vitro.

TABLE 1
Measured K_{b} at hCGRP for Examples 1-3.

| Example | | |
|---|---|---|
| Number | hCGRP Kb ± SD (nM) | n |
| 1 | 0.08 ± 0.01 | 8 |
| 2 | 0.14 ± 0.02 | 13 |
| 3 | 0.10 ± 0.02 | 3 |

### Characterization of Compound Binding Affinity for CGRP1 Receptors Using Membranes Prepared from SK-N-MC Neuroblastoma Cells

The equilibrium affinity constant (Ki) at the human CGRP receptor heterodimer may be determined using standard competition filtration binding methods with membranes prepared from cultured SK-N-MC neuroblastoma cells (ATCC) and a high-affinity CGRP receptor antagonist.

Membrane Production: Human SK-N-MC neuroblastoma cells, endogenously expressing the human CGRP receptor, are expanded in culture flasks (Corning, T225) using growth media containing Eagles's Minimum Essential Media (HyClone™) supplemented with 10% heat-inactivated fetal bovine serum (GIBCO®). When cell monolayers achieve a level of 70-80% confluence, they are dissociated from the culture flasks using TrypLE™ cell dissociation media (ThermoFisher Scientific). Dissociated cells are pelleted by centrifugation at 300×g to remove the growth and dissociation media. Centrifuged cell pellets are flash frozen in liquid nitrogen (−320° F.) for 30 sec and stored frozen at −80° C. until the subsequent membrane preparation.

A membrane preparation P2 pellet (second pellet from centrifugation procedure) is generated from harvested SK-N-MC frozen cultured cell pellets by diluting these cells on ice into 20 mM Tris-HCl buffer, pH 7.4, containing protease inhibitors (Pierce). Suspended cells are Dounce homogenized on ice and centrifuged at low speed (1000×g for 20 min at 4° C.) to remove cellular organelles and debris (P1 pellet). The supernatant, containing the soluble membrane fraction, is collected and subjected to a high speed centrifugation step (25,000×g for 1 hr at 4° C.) to isolate the resultant P2 membrane pellet. This P2 centrifugation pellet is suspended in buffer containing 20 mM Tris-HCl, 1 mM EDTA, and 1 mM MgCl₂, at a pH of 7.4, to obtain a final protein concentration of 3.8 mg protein/mL. Protein concentration is determined using the Bradford Protein Assay (ThermoFisher Scientific). Aliquoted P2 membrane preparations are flash frozen in liquid nitrogen (−320° F.) for 30 sec and stored in ultra-low freezer (−80° C.) until use in the binding assay.

Binding Affinity Characterization: Test compounds are dissolved in DMSO to a concentration of 10 mM and diluted to 400 nM (100 nM final concentration) in assay buffer. Compounds are serially diluted in assay buffer to obtain an 11-point concentration response stock dilution plate. The stock 11-point dilution plate is subsequently stamped into the assay plate (62.5 μL) at a concentration 4× higher than the final compound test concentration. [³H]BIBN-4096 (see V. P. Shevchenko, I. Yu. Nagaev, N. F. Myasoedov. A. B. Susan, K.-H. Switek, and H. Braunger, J Label Compd Radiopharm 2006; 49: 421-427) is diluted to a 4× stock concentration of 520 pM (target 130 pM/well, final concentration). The binding assay is initiated through the addition of [³H]BIBN-4096 radioligand (in 62.5 μL assay buffer) to serially diluted test compound and 50 μL of SK-N-MC membrane (in assay buffer; 20 μg/well). Total assay volume is 250 μL/well. After an incubation period of 60 min, the reaction is terminated by transferring 200 μL to a GF/B Whatman (Millipore), which is pretreated for 60 min with 0.3% polyethylenimine (PEI) and washed three times in ice-cold 50 mM Tris-HCl, pH 7.4, using a 405 TS plate washer (BioTek®). The plate is subsequently washed three times with ice-cold 50 mM Tris-HCl buffer at pH 7.4. Plates are dried overnight. Emulsifier-Safe™ (PerkinElmer) is added to the filtration plates (100 μL/well).

Data Analysis: Bound radioactivity may be counted using a MicroBeta® Trilux Scintillation Counter (PerkinElmer®). Specific binding is defined as counts which are displaceable by 10 μM BIBN-4096 (MCE® MedChemExpress). Relative IC₅₀ values are calculated using a four-parameter logistic curve fitting program (GraphPad Prism v8.3.0). Equilibrium dissociation binding constants (Kᵢ) for the compounds are calculated from the following equation:

Kᵢ=IC₅₀/(1+[L]/K_{d})

where IC₅₀=the concentration of compound resulting in 50% inhibition of binding activity, [L]=the radioligand concentration for the experiment, K_{d}=equilibrium dissociation constant for the radioligand determined from saturation binding analysis. Calculated Kᵢ values are reported as means and SEM.

Results: This binding affinity characterization procedure is used to define the Kᵢ for each of the Examples 1-3, which are summarized below in Table 2. Each of these three compounds demonstrates a high affinity specific binding for human CGRP receptor heterodimer.

TABLE 2
CGRP Binding Affinity Results

| Example | | | |
|---|---|---|---|
| Number | Ki, nM | SEM | n |
| 1 | 0.35 | 0.10 | 5 |
| 2 | 0.98 | 0.24 | 5 |
| 3 | 0.93 | 0.36 | 3 |

Ki values are means, expressed to 3 significant digits.
Standard error of the mean (SEM) is expressed with the same number of decimal places as the mean, n is the number of times tested.

### In Vitro Determination of Efflux by ABCB1, Human P-Glycoprotein (Pgp)

Cell Culture: MDCKII cells stably expressing human wild-type ABCB1 (Pgp) are obtained from the Netherlands Cancer Institute (Amsterdam, The Netherlands). MDCK cells are maintained as described previously (Desai et al., Mol Pharm 10:1249-1261, 2013).

Bi-directional transport across MDCK cells: The assay is essentially conducted as described previously (Desai et al., Mol Pharm 10:1249-1261, 2013). Transport is measured in both directions across uninhibited and inhibited cell monolayers using a substrate concentration of 5 μM diluted from a 10 mM DMSO stock solution (final DMSO concentration of 0.05%) and a single 60-min time interval. 2.5 μM of the compound of Example 1 is used to selectively inhibit Pgp. The apparent permeability coefficients (Papp) are estimated as the slope of the mass transported per 60 min relative to the total recovered mass. The basal-to-apical (B-A)/apical-to-basal (A-B) Papp ratios are calculated in the absence or presence of inhibitor in each cell line for net efflux ratio (NER).

Result: The NER of the compound of Example 1 for efflux by Pgp is determined to be 1.9, and for Example 2, the NER for efflux by Pgp is determined to be 1.6.

### In Vivo Rat Tracer Distribution and Kinetics Studies

### Tracer Distribution and Brain Uptake in Rats

Tracer Mix Instructions

Stock Formulation: Prepare a tracer stock solution at 0.5 mg/mL in 25% HP-BCD/PW (corrected for salt weight). Vortex thoroughly for 30 sec and place in bath sonication for 30 mins. Confirm the stock formulation is a clear solution or homogeneous suspension. Acid (10 μL acetic acid) or base (10 μL 5N NaOH), probe sonication, or a sonic bath may be used to aid in solubilization.

Final Dosing Formulation: If the stock formulation is a solution or homogeneous suspension, allow the stock solution to sit at room temperature for 5 min, and confirm and document the appearance of the stock formulation. Dilute the stock solution to the appropriate dose concentrations with 25% HP-BCD/PW. Vortex the final dosing solutions for 30 sec. Final tracer dosing solution is used for generation of the LC/MS/MS calibration standards in the appropriate matrix.

Study populations: Animal studies are performed under protocols approved by Eli Lilly and Company and PreClinOmics Institutional Animal Care and Use Committee. Twenty Sprague-Dawley rats weighing 200-300 g are obtained from Harlan Sprague Dawley Inc. (Indianapolis, Ind.) and are randomized into 4 groups of 5 animals each. Animals have access to food and water ad libitum before the study.

Live Phase Methods: Five animals per dose group are used. Each animal receives 10 μg/kg of the CGRP tracer compound administered intravenously in the lateral tail vein. Animals are euthanized by cervical dislocation followed by decapitation or live decapitation after 5, 10, 20 or 40 min survival intervals.

Trunk blood is collected in EDTA-coated Eppendorf tubes and stored on wet ice until study completion. The whole brain is rapidly removed, and lightly rinsed with sterile water. Frontal cortex, hippocampus, cerebellum, brainstem, and striatum brain tissues are dissected, weighed, stored in 1.5 mL Eppendorf tubes, and placed on wet ice until completion of live phase study.

Using a drug naïve test subject, blood and seven cortical brain tissues samples are collected for use in generating blank and standard curve samples.

Tracer Extraction & Sample Preparation Method

Tissue—Tissue samples are kept on wet ice until the completion of the live phase. Tissue samples collected from test and naïve subjects are homogenized at PreClinOmics (Indianapolis, Ind., USA) and immediately taken to AIT Bioscience (Indianapolis, Ind., USA) on wet ice to be centrifuged along with an excel sheet indicating tissue weights, amount of ACN+0.1% HCOOH added to all tissue samples, and how the naive tissues were spiked for standards. Details on processing at PreClinOmics are as follows:

ACN containing 0.1% HCOOH is added to each tissue sample at a volume of four times the weight of the tissue sample (e.g., Add 600 μL ACN to a 150 mg brain tissue). The sample is homogenized via probe sonication. The standard curve is a 6-point curve with the range of 0.3-60 ng/g, linear regression, correlation coefficient, R², minimum of ≥0.95. Calibration standards are prepared from control tissue matrix homogenate (of the same organ) spiked with a calculated volume of standard (prepared from the tracer dosing solution used during the live phase). The homogenized tissue samples and spiked standards are transferred to AIT Bioscience on wet ice. Once at AIT Biosciences, all homogenized samples are centrifuged for 20 min at 14,000 rpm. The supernatant solution is diluted with an internal standard solution (1.0 ng/mL diphenhydramine in water) at a ratio of 1:4. Following dilution of the supernatant, if the total sample volume in the well plate is too small for injection, an additional dilution is performed using the mobile phase, ACN:water:0.1% HCOOH (20:80:0.1), at a ratio of 1:1. This solution is mixed thoroughly and analyzed via LC/MS/MS for tracer compound. Additional dilutions incurred due to small sample volume have an appropriate dilution factor applied during regression analysis.

Blood Samples—Whole blood samples are kept on wet ice until the completion of the live phase. Centrifuge blood samples at 14,000 rpm for 20 min. After centrifugation, plasma samples are transferred to labeled tubes, stored on wet ice, and delivered to AIT Bioscience (along with tissue samples) for analysis of blocker test article. 200 μL of ACN containing 0.1% HCOOH is added to each 50 μL plasma sample. Samples are placed in an ultrasonic water bath for 5 min, followed by centrifugation at 14,000 rpm for 20 min. Standard curve is a 6-point curve with the range of 0.1-30 ng/mL with linear regression correlation coefficient, R², minimum of ≥0.95. Calibration standards are prepared from control plasma spiked with a calculated volume of standard (prepared from the tracer dosing solution used during the live phase). The supernatant solution is diluted with an internal standard solution (1.0 ng/mL diphenhydramine in water) at a ratio of 1:3. This solution is then mixed thoroughly and analyzed via LC/MS/MS for tracer compound.

| | |
|---|---|
| Organic Solvent for Small | Acetonitrile + 0.1% formic acid |
| Molecule Extraction | |
| Amount of Organic Solvent | 4x Tissue Weight (volume in |
| to Tissue | μL) |
| Amount of Organic Solvent | 200 μL ACN:50 μL plasma |
| to Plasma | |
| Supernatant Dilution with | 1 volume tissue supernatant (in |
| Internal Standard Solution | μL) : 4 volumes sterile internal |
| (1.0 ng/mL diphenhydramine | standard solution (in μL) |
| in water) | |
| Supernatant Dilution with | 1 volume plasma supernatant |
| Internal Standard Solution | (in μL): 3 volumes internal |
| (1.0 ng/mL diphenhydramine | standard solution (in pL) |
| in water) | |
| Organic Solvent for Dilution | ACN:Water:HCOOH |
| of Small Sample | |
| Volumes (optional based | (20:80:0.1) |
| on aliquot volume) | |

LC-MS/MS Parameters

LC-MS/MS analysis of tracer concentration in brain tissue is accomplished using a model DIONEX™ ULTIMATE™ 3000 LC auto sampler (Thermo Fisher Scientific, MA USA) linked to a THERMO SCIENTIFIC™ TSQ QUANTIVA™ triple quadruple mass spectrometer (Thermo Fisher Scientific, MA USA). A 20 μL injection of the sample solution is made onto a Waters BEH C18 column (2.1 mm×50 mm; 1.7 μm; part #176000863) maintained at 25-30° C. using a mixture of ACN:water: 0.1% HCOOH as mobile phase, at a flow rate of 0.4 mL/min. The mixture of ACN:water varies in order to have the analyte retention time stay within a range of 1-6 minutes (based on LC conditions). Tracer eluting from the column is identified by its characteristic retention time and mass to charge (m/z) ratio, and quantified by comparison to a standard curve prepared in appropriate tissue matrix. Tracer levels in tissue are represented in units of ng/g of tissue. Tracer levels in plasma are represented in units of ng/ml of plasma.

Example 1—The precursor to product ion transition monitored is Q1=412.32, Q3=117.229. An isocratic method is used lasting 2 min with a retention time of 1.1 min. The mobile phase consists of water (72.5%) and ACN (27.5%) with 0.1% HCOOH.

Example 2—The precursor to product ion transition monitored is Q1=412.161, Q3=230.224. A gradient method is used lasting 2.5 min with a retention time of 1.2 min. The mobile phase consists of various ratios of water and ACN with 0.1% HCOOH. The gradient conditions are 73% water and 27% ACN from 0-1.7 min, gradient to 10% water and 90% ACN from 1.75 min-2.3 min, and 73% water 27% B from 2.35 minutes-2.5 min.

Example 3—The precursor to product ion transition monitored is Q1=430.27, Q3=248.117. An isocratic method is used lasting 2.3 min with a retention time of 1.1 min. The mobile phase consists of water (72.5%) and ACN (27.5%) with 0.1% HCOOH.

Statistical Analysis: Tracer distribution is summarized by treatment group mean tracer concentration (ng/g or ng/ml)±SEM.

The tracer distribution and brain uptake data in rat for Examples 1-3 are displayed in Tables 3-5.

TABLE 3
Example 1 mean tracer concentrations ± SEM (ng/g) in brain tissue region (ng/mL) in plasma

| | Example 1 | | | | | |
|---|---|---|---|---|---|---|
| | | | | | | Mean tracer conc |
| Time | | | | | | (plasma) ± SEM |
| point | Mean tracer concentrations ± SEM (ng/g) by Tissue Region | | | | | (ng/mL) |
| (min) | Frontal Cortex | Hippocampus | Cerebellum | Brain Stem | Striatum | Plasma |
| 5 | 4.19 ± 0.29 | 3.52 ± 0.32 | 3.52 ± 0.16 | 3.69 ± 0.29 | 3.94 ± 0.32 | 6.3 ± 0.63 |
| 10 | 2.27 ± 0.05 | 2.33 ± 0.09 | 1.91 ± 0.05 | 2.46 ± 0.09 | 2.44 ± 0.13 | 3.37 ± 0.37 |
| 20 | 0.98 ± 0.06 | 1.06 ± 0.07 | 0.9 ± 0.06 | 1.21 ± 0.08 | 1.12 ± 0.07 | 1.72 ± 0.12 |
| 40 | 0.29 ± 0.02 | 0.33 ± 0.02 | 0.28 ± 0.02 | 0.41 ± 0.03 | 0.34 ± 0.02 | 0.48 ± 0.05 |

TABLE 4
Example 2 mean tracer concentrations ± SEM (ng/g) in brain tissue region (ng/mL) in plasma

| | Example 2 | | | | | |
|---|---|---|---|---|---|---|
| | | | | | | Mean tracer conc |
| Time | | | | | | (plasma) ± SEM |
| point | Mean tracer concentrations ± SEM (ng/g) by Tissue Region | | | | | (ng/mL) |
| (min) | Frontal Cortex | Hippocampus | Cerebellum | Brain Stem | Striatum | Plasma |
| 5 | 9.48 ± 0.57 | 7.66 ± 0.42 | 8.76 ± 0.48 | 8.73 ± 0.51 | 9.36 ± 0.54 | 9.82 ± 0.82 |
| 10 | 5.07 ± 0.56 | 4.64 ± 0.78 | 4.78 ± 0.78 | 5.27 ± 0.85 | 5.23 ± 0.91 | 5.54 ± 0.8 |
| 20 | 2.19 ± 0.24 | 2.29 ± 0.36 | 2.08 ± 0.22 | 2.56 ± 0.33 | 2.42 ± 0.28 | 2.88 ± 0.37 |
| 40 | 0.8 ± 0.09 | 0.74 ± 0.14 | 0.88 ± 0.11 | 1.08 ± 0.08 | 1.06 ± 0.16 | 1.03 ± 0.12 |

TABLE 5
Example 3 mean tracer concentrations ± SEM (ng/g) in brain tissue region (ng/mL) in plasma

| | Example 3 | | | | | |
|---|---|---|---|---|---|---|
| | | | | | | Mean tracer conc |
| Time | | | | | | (plasma) ± SEM |
| point | Mean tracer concentrations ± SEM (ng/g) by Tissue Region | | | | | (ng/mL) |
| (min) | Frontal Cortex | Hippocampus | Cerebellum | Brain Stem | Striatum | Plasma |
| 5 | 3.55 ± 0.33 | 2.9 ± 0.2 | 3.06 ± 0.22 | 3.13 ± 0.22 | 3.57 ± 0.28 | 6.2 ± 0.5 |
| 10 | 1.92 ± 0.16 | 1.91 ± 0.14 | 1.76 ± 0.13 | 2.01 ± 0.17 | 2.14 ± 0.16 | 3.65 ± 0.26 |
| 20 | 0.92 ± 0.05 | 1.01 ± 0.06 | 0.88 ± 0.06 | 1.18 ± 0.06 | 1.09 ± 0.07 | 1.94 ± 0.15 |
| 40 | 0.31 ± 0.02 | 0.34 ± 0.02 | 0.31 ± 0.02 | 0.42 ± 0.02 | 0.36 ± 0.04 | 0.73 ± 0.04 |

The data of Tables 3-5 indicates that, at tracer doses of 10 μg/kg, the compounds of Examples 1-3 are brain penetrant, essentially evenly distributed throughout brain tissue, and maintain a constant B/P ratio over time in the rat species.

We claim:

1. A compound of the formula: 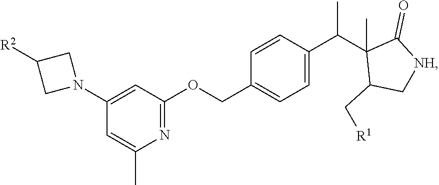
wherein R¹ is hydrogen, F, or ¹⁸F; and
R² is hydrogen, F, or ¹⁸F;
or a pharmaceutically acceptable salt thereof,
provided that when R¹ is ¹⁸F then R² is not ¹⁸F.

2. The compound according to claim 1 wherein the compound is of the formula: 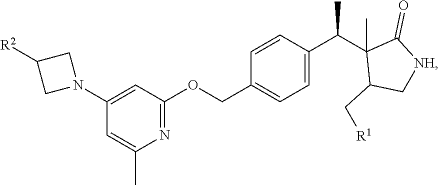
or a pharmaceutically acceptable salt thereof,
provided that when R¹ is ¹⁸F then R² is not ¹⁸F.

3. The compound according claim 1 wherein the compound is of the formula: 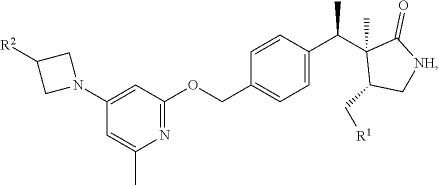
or a pharmaceutically acceptable salt thereof,
provided that when R¹ is ¹⁸F then R² is not ¹⁸F.

4. The compound according to claim 1 wherein R¹ is ¹⁸F or F and R² is hydrogen, or a pharmaceutically acceptable salt thereof.

5. The compound according to claim 1 wherein R² is ¹⁸F or F and R¹ is hydrogen, or a pharmaceutically acceptable salt thereof.

6. The compound according to claim 1 wherein the compound is selected from the group consisting of: 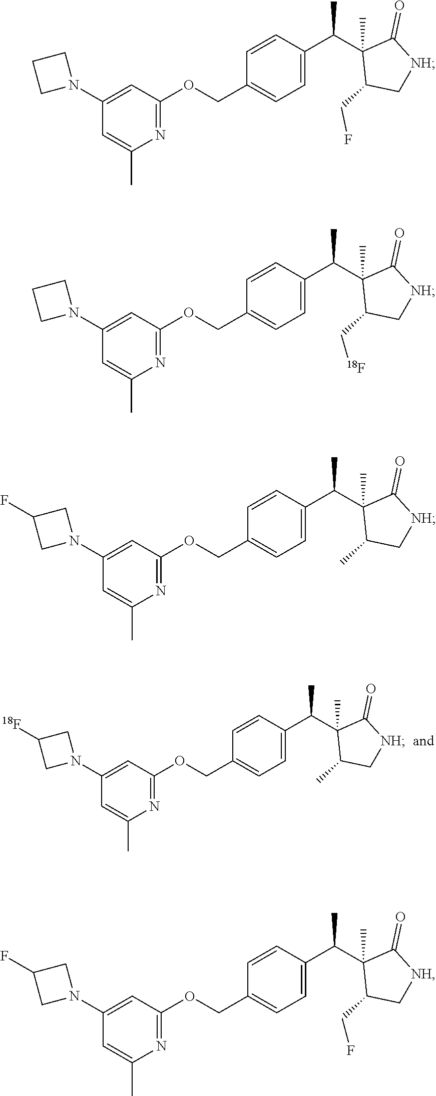 [SMILES: Cc1cc(N2CCC2)cc(OCc2ccc([C@@H](C)[C@@]3(C)C(=O)NC[C@@H]3CF)cc2)n1 ; Cc1cc(N2CCC2)cc(OCc2ccc([C@@H](C)[C@@]3(C)C(=O)NC[C@@H]3C[18F])cc2)n1 ; Cc1cc(N2CC(F)C2)cc(OCc2ccc([C@@H](C)[C@@]3(C)C(=O)NC[C@@H]3C)cc2)n1 ; Cc1cc(N2CC([18F])C2)cc(OCc2ccc([C@@H](C)[C@@]3(C)C(=O)NC[C@@H]3C)cc2)n1 ; Cc1cc(N2CC(F)C2)cc(OCc2ccc([C@@H](C)[C@@]3(C)C(=O)NC[C@@H]3CF)cc2)n1]
or a pharmaceutically acceptable salt thereof.

7. The compound according to claim 1 wherein the compound is: 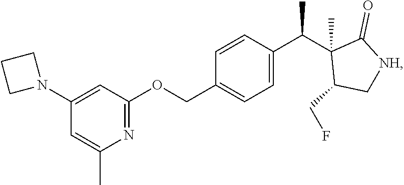 [SMILES: Cc1cc(N2CCC2)cc(OCc2ccc([C@@H](C)[C@@]3(C)C(=O)NC[C@@H]3CF)cc2)n1]
or a pharmaceutically acceptable salt thereof.

8. The compound according to claim 1 wherein the compound is: 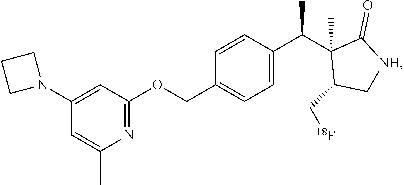 [SMILES: Cc1cc(N2CCC2)cc(OCc2ccc([C@@H](C)[C@@]3(C)C(=O)NC[C@@H]3C[18F])cc2)n1]
or a pharmaceutically acceptable salt thereof.

9. The compound according to claim 1 wherein the compound is: 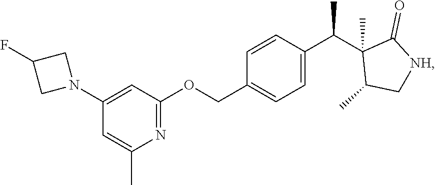 [SMILES: Cc1cc(N2CC(F)C2)cc(OCc2ccc([C@@H](C)[C@@]3(C)C(=O)NC[C@@H]3C)cc2)n1]
or a pharmaceutically acceptable salt thereof.

10. The compound according to claim 1 wherein the compound is: 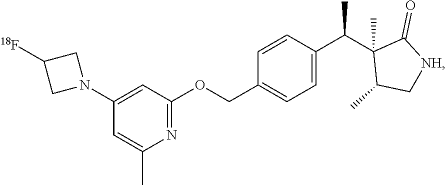 [SMILES: Cc1cc(N2CC([18F])C2)cc(OCc2ccc([C@@H](C)[C@@]3(C)C(=O)NC[C@@H]3C)cc2)n1]
or a pharmaceutically acceptable salt thereof.

11. A compound of the Formula: 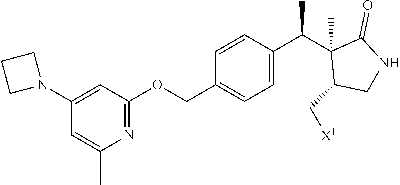
wherein X¹ is a suitable leaving group.

12. The compound according to claim 11 wherein the suitable leaving group is methanesulfonyl or 4-methylbenzenesulfonyl.

13. A compound of the Formula: 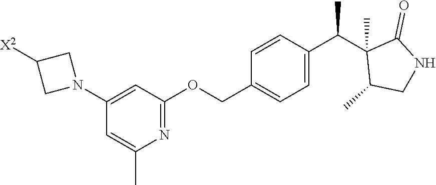
wherein X² is a suitable leaving group.

14. The compound according to claim 13 wherein the suitable leaving group is methanesulfonyl or 4-methylbenzenesulfonyl.

15. A compound of the Formula: 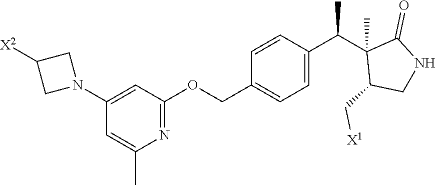
wherein X¹ and X² are each independently suitable leaving groups.

16. The compound according to claim 15 wherein the suitable leaving groups are each independently selected from the group consisting of methanesulfonyl and 4-methylbenzenesulfonyl.

17. A pharmaceutical composition, comprising a compound or salt according to claim 1 and one or more pharmaceutically acceptable carriers, diluents, or excipients.

18. A method of using a radiolabeled compound of the Formula: 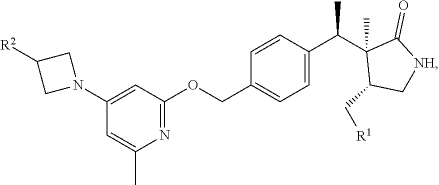
wherein R¹ is hydrogen or ¹⁸F; and
R² is hydrogen or ¹⁸F; or a pharmaceutically acceptable salt thereof, provided that when R¹ is ¹⁸F then R² is not ¹⁸F,
comprising introducing into a mammal a detectable quantity of the radiolabeled compound, allowing sufficient time for the radiolabeled compound to become associated with CGRP receptors in the brain of the mammal, and then detecting the radiolabeled compound in the brain of the mammal.

19. The method of claim 18 wherein the radiolabeled compound is detected using positron emission tomography.

20. A method of preparing a radiolabeled compound of the Formula: 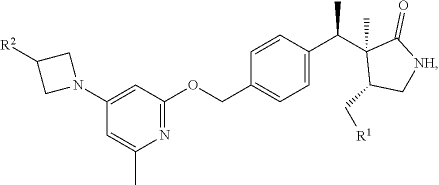
wherein R¹ is hydrogen or ¹⁸F; and
R² is hydrogen or ¹⁸F, provided that when R¹ is ¹⁸F then R² is not ¹⁸F,
the method comprising reacting a compound of the Formula: 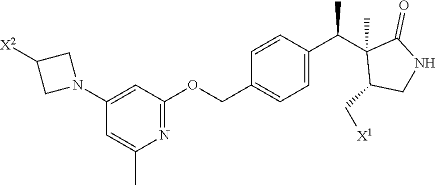
with a source of [¹⁸F]fluoride,
wherein X¹ is hydrogen or a suitable leaving group; and
X² is hydrogen or a suitable leaving group.

21. The method according to claim 20 wherein X¹ is a suitable leaving group and X² is hydrogen.

22. The method according to claim 20 wherein X¹ is hydrogen and X² is a suitable leaving group.

23. The method according to claim 20 wherein X¹ and X² are each independently suitable leaving groups.
